(19) United States
(12) Patent Application Publication
(10) Pub. No.: US 2024/0382507 A1
(43) Pub. Date: Nov. 21, 2024

(54) PHARMACEUTICAL COMPOSITIONS COMPRISING 2,3,4,5- TETRAHYDROBENZOTHIEPIN- 1,1-DIOXIDE DERIVATIVES AND THE USE THEREOF

(71) Applicant: Mirum Pharmaceuticals, Inc., Foster City, CA (US)

(72) Inventor: Parag VED, Foster City, CA (US)

(73) Assignee: Mirum Pharmaceuticals, Inc., Foster City, CA (US)

(21) Appl. No.: 18/667,354

(22) Filed: May 17, 2024

Related U.S. Application Data
(60) Provisional application No. 63/467,823, filed on May 19, 2023.

(51) Int. Cl. A61K 31/7042; A61K 9/48

(52) U.S. Cl. CPC A61K 31/7042; A61K 9/485; A61K 9/4858; A61K 9/4866

(57) ABSTRACT

Provided herein are solid dosage forms comprising 2,3,4,5-tetrahydro-benzothiepin-1,1-dioxide derivatives, for example volixibat or a pharmaceutically acceptable salt thereof. Also provided herein are embodiments directed to methods of treating cholestatic liver disease, hyperlipidemia, arteriosclerosis, or Syndrome X, or lowering the serum cholesterol level in a subject in need thereof, wherein the methods comprise administering to the subject a therapeutically effective amount of the solid dosage form of the pharmaceutical composition comprising volixibat.

### CROSS REFERENCE TO RELATED APPLICATIONS

This application claims the benefit under 35 U.S.C. § 119(e) of U.S. Provisional Application No. 63/467,823, filed May 19, 2023, which is incorporated herein by reference in its entirety.

### FIELD

Provided herein are solid dosage forms comprising 2,3,4,5-tetrahydro-benzothiepin-1,1-dioxide derivatives, for example, volixibat or its pharmaceutically acceptable salt thereof.

### BACKGROUND

Hypercholemia and cholestatic liver diseases are liver diseases associated with impaired bile secretion (i.e., cholestasis), associated with and often secondary to the intracellular accumulation of bile acids/salts in the hepatocyte. Hypercholemia is characterized by increased serum concentration of bile acid or bile salt. Cholestasis can be categorized clinicopathologically into two principal categories of obstructive, often extrahepatic, cholestasis, and nonobstructive, or intrahepatic, cholestasis. Nonobstructive intrahepatic cholestasis can further be classified into two principal subgroups of primary intrahepatic cholestasis that result from constitutively defective bile secretion, and secondary intrahepatic cholestasis that result from hepatocellular injury. Primary intrahepatic cholestasis includes diseases such as benign recurrent intrahepatic cholestasis, which is predominantly an adult form with similar clinical symptoms, and progressive familial intrahepatic cholestasis (PFIC) types 1, 2, and 3, which are diseases that affect children. Pediatric cholestatic liver diseases affect a small percentage of children, but therapy results in significant healthcare costs each year. Currently, many of the pediatric cholestatic liver diseases require invasive and costly treatments such as liver transplantation and surgery.

Volixibat, also known as SHP626, LUM002, or ((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate), is an ileal bile acid transporter (IBAT) inhibitor to treat hypercholemia and cholestatic liver diseases. There remains a need to discover proper dosage forms of volixibat having favorable dissolution and pharmacokinetic profiles, which also demonstrate good storage stability.

### SUMMARY

Provided herein are embodiments directed to a pharmaceutical composition comprising a compound of Formula (I),

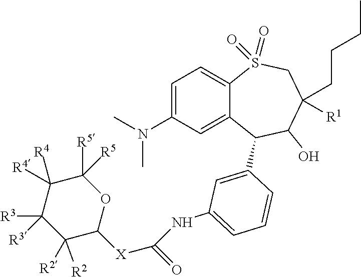

wherein:
X is NH;
R¹ is (C₁-C₆)-alkyl;
R² is OH;
R^{2′} is H;
R³, R^{3′}, R⁴, R^{4′}, R⁵, R^{5′}, are, independently H, Cl, Br, I, OH, —(CH₂)—OH, CF₃, NO₂, N₃, CN, S(O)ₚ—R⁶, O—S(O)ₚ—R⁶, (C₁-C₆)-alkylene-S(O)ₚ—R⁶, (C₁-C₆)-alkylene-O—S(O)ₚ—R⁶, COOH, COO(C₁-C₆)alkyl, CONH₂, CONH(C₁-C₆)alkyl, CON[(C₁-C₆)alkyl]2, (C₁-C₆)-alkyl, (C₂-C₆)alkenyl, (C₂-C₆)-alkynyl, O—(C₁-C₆)-alkyl, where one, more than one, or all hydrogen(s) in the alkyl radicals may be replaced by fluorine; phenyl, (CH₂)-phenyl, (CH₂)₂-phenyl, O-phenyl, O—(CH₂)ₘ-phenyl, —(CH₂)—O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂; wherein at least one of the radicals R³, R^{3′}, R⁴, R^{4′}, R⁵, and R^{5′} has the meaning of (C₁-C₆)-alkylene-O—S(O)ₚ—R6 and another has the meaning of —O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂;
R⁶ is H, OH, (C₁-C₆)-alkyl, NH₂, NH(C₁-C₆)-alkyl or N((C₁-C₆)-alkyl)₂;
n is 2, 3, 4, 5 or 6;
m is 1, 2, 3, 4, 5 or 6;
p is 0, 1 or 2;
or a pharmaceutically acceptable salt thereof, and one or more excipients selected from the group consisting of:
   (i) a diluent or a filler,
   (ii) a disintegrant,
   (iii) a channeling agent,
   (iv) a glidant, and
   (v) a lubricant.

In some embodiments, the compound of Formula (I) has the structure of Formula (II) that is volixibat:

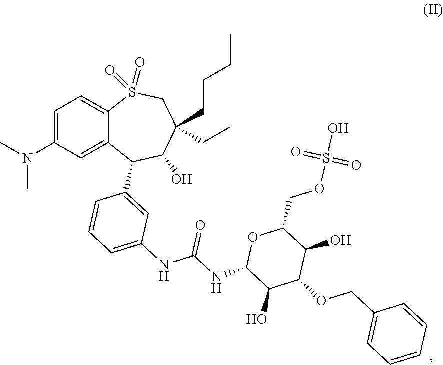 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O]

or a pharmaceutically acceptable salt thereof.

Also provided herein are embodiments directed to methods of treating cholestatic liver disease, hyperlipidemia, arteriosclerosis, or Syndrome X, or lowering the serum cholesterol level in a subject in need thereof, wherein the methods comprise administering to the subject a therapeutically effective amount of the pharmaceutical composition as described herein.

Further provided herein are embodiments directed to a method of producing the solid form pharmaceutical composition comprising the steps of: combining a therapeutically effective amount of the compound of Formula (I) and one or more excipients selected from the group consisting of:
(i) a diluent or a filler,
(ii) a disintegrant,
(iii) a channeling agent,
(iv) a glidant, and
(v) a lubricant
   to form an admixture; mixing the admixture; and filling in a capsule shell or compacting the admixture to form the pharmaceutical composition.

Additionally provided herein are embodiments directed to a kit for treating cholestatic liver disease or hyperlipidemia in a subject in need thereof, wherein the kit comprises a therapeutically effective amount of the composition as described herein.

### BRIEF DESCRIPTION OF THE DRAWINGS

FIG. 1 shows NaCl:volixibat sample agglomeration.

FIG. 2 shows PEG 1500:volixibat sample agglomeration.

FIG. 3 shows ambient/60% RH condition sample appearance.

FIG. 4 shows 40° C./75% RH condition sample appearance.

FIG. 5 shows dissolution data for Formulations 1 and 2, 20 mg capsules in 900 mL phosphate pH 6.8.

FIG. 6 shows dissolution data for Formulations 1, 3, and 4, 40 mg capsules in 500 mL phosphate buffer pH 6.8.

FIG. 7 shows dissolution data for Formulation 5, 20 mg capsule: T=0 tamped vs bulk fill.

FIG. 8 shows dissolution data for Formulation 5, 20 mg capsule: effect of over-lubrication.

FIG. 9 shows dissolution data for Formulation 5, 20 mg capsules in 500 mL acetate pH 4.5.

FIG. 10 shows dissolution data for Formulations 1 and 3-6, 20 mg capsules in 500 mL phosphate pH 6.8.

FIG. 11 shows dissolution data for Formulations 1 and 3-6, 20 mg capsules in 500 mL 0.01N HCl.

FIG. 12 shows dissolution data for Formulation 5, 20 mg capsules in each dissolution media.

FIG. 13 shows dissolution data for Formulation 7, 20 mg capsules in each dissolution media.

### DETAILED DESCRIPTION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

As used herein, the term “active pharmaceutical ingredient” or “API” refers to a biologically active compound. Exemplary APIs include volixibat. Volixibat is known as SHP626, LUM002, or ((2R,3R,4S,5R,6R)-4-benzyloxy-6-{3-[3-((3S,4R,5R)-3-butyl-7-dimethylamino-3-ethyl-4-hydroxy-1,1-dioxo-2,3,4,5-tetrahydro-1H-benzo[b]thiepin-5-yl)-phenyl]-ureido}-3,5-dihydroxy-tetrahydro-pyran-2-ylmethyl) hydrogen sulfate). The structure of volixibat potassium is shown below:

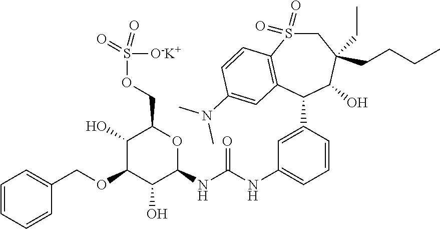

The terms “alkoxy”, “phenyloxy”, “benzoxy” and “pyrimidinyloxy” refer to an alkyl group, phenyl group, benzyl group, or pyrimidinyl group, respectively, each optionally substituted, that is bonded through an oxygen atom. For example, the term “alkoxy” means a straight or branched —O-alkyl group of 1 to 20 carbon atoms, including, but not limited to, methoxy, ethoxy, n-propoxy, isopropoxy, t-butoxy, and the like. In some embodiments, the alkoxy chain is from 1 to 10 carbon atoms in length, from 1 to 8 carbon atoms in length, from 1 to 6 carbon atoms in length, from 1 to 4 carbon atoms in length, from 2 to 10 carbon atoms in length, from 2 to 8 carbon atoms in length, from 2 to 6 carbon atoms in length, or from 2 to 4 carbon atoms in length.

As used herein, the term “alkyl” means a saturated hydrocarbon group which is straight-chained or branched. An alkyl group can contain from 1 to 20, from 2 to 20, from 1 to 10, from 2 to 10, from 1 to 8, from 2 to 8, from 1 to 6, from 2 to 6, from 1 to 4, from 2 to 4, from 1 to 3, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylene” or “alkylenyl” means a divalent alkyl linking group. An example of an alkylene (or alkylenyl) is methylene or methylenyl (—CH₂—).

As used herein, the term “alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

As used herein, the term “amino” means —NH₂.

As used herein, the term “aminoalkoxy” means an alkoxy group substituted by an amino group. An example of an aminoalkoxy is —OCH₂CH₂NH₂.

As used herein, the term “aminoalkyl” means an alkyl group substituted by an amino group. An example of an aminoalkyl is —CH₂CH₂NH₂.

As used herein, the term “aminosulfonyl” means —S(═O)₂NH₂.

As used herein, the term “aminoalkylthio” means an alkylthio group substituted by an amino group. An example of an aminoalkylthio is —SCH₂CH₂NH₂.

As used herein, the term “amphiphilic” means a three-dimensional structure having discrete hydrophobic and hydrophilic regions. An amphiphilic compound suitably has the presence of both hydrophobic and hydrophilic elements.

As used herein, the term “animal” includes, but is not limited to, humans and non-human vertebrates such as wild, domestic, and farm animals.

As used herein, the term “aryl” means a monocyclic, bicyclic, or polycyclic (e.g., having 2, 3 or 4 fused rings) aromatic hydrocarbons optionally comprising heteroatoms. In some embodiments, aryl groups have from 6 to 20 carbon atoms or from 6 to 10 carbon atoms. Examples of aryl groups include, but are not limited to, phenyl, naphthyl, anthracenyl, phenanthrenyl, indanyl, indenyl, tetrahydronaphthyl, and the like. In some embodiments, aryl groups include heteroaryl groups defined in other embodiments. In some embodiments, aryl groups have heteroatoms. In some embodiments, aryl groups have heteroatoms selected from the group consisting of N, S, O, and P atoms. Examples of aryl groups include, but are not limited to:

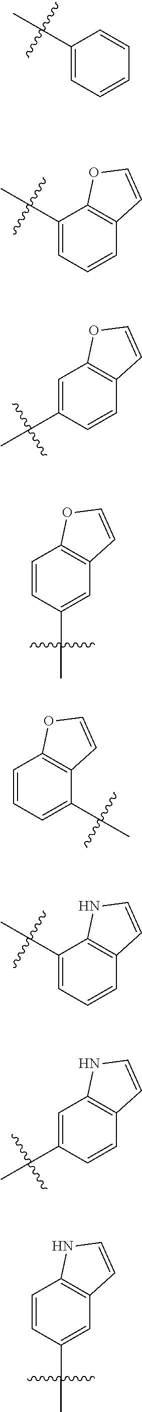

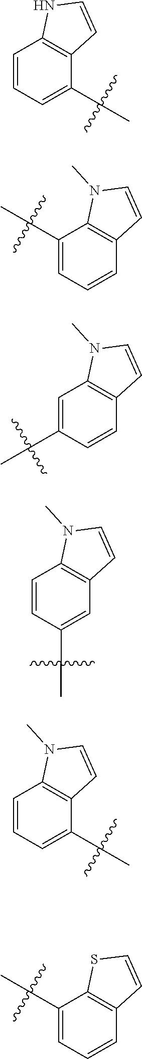

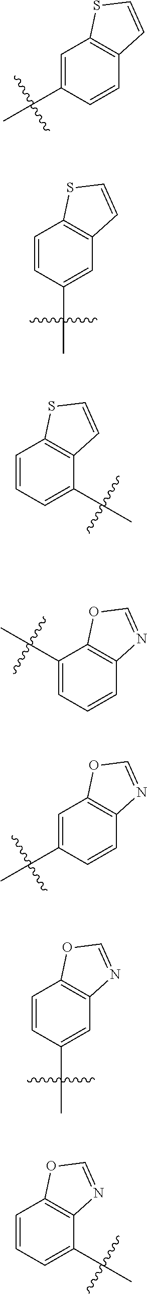

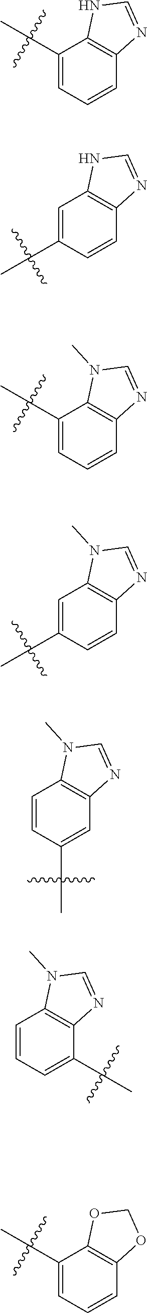

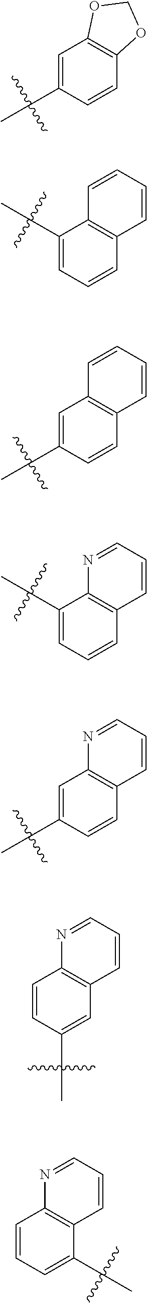

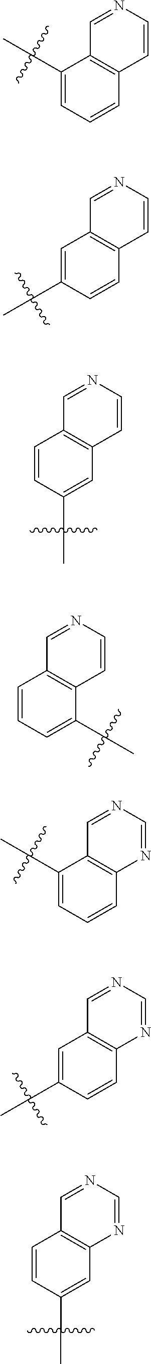

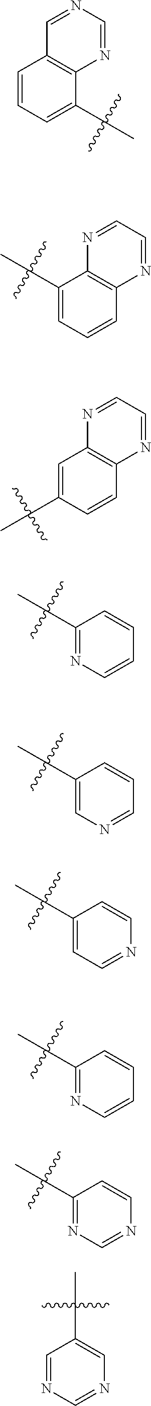

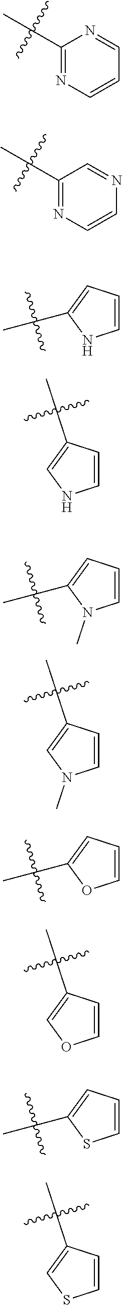

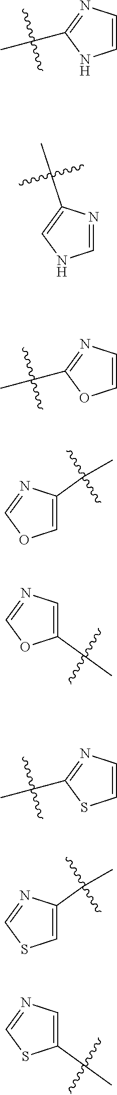

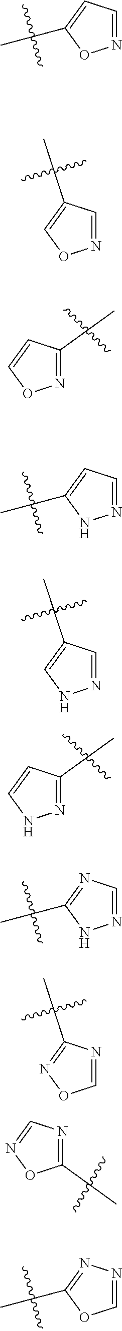

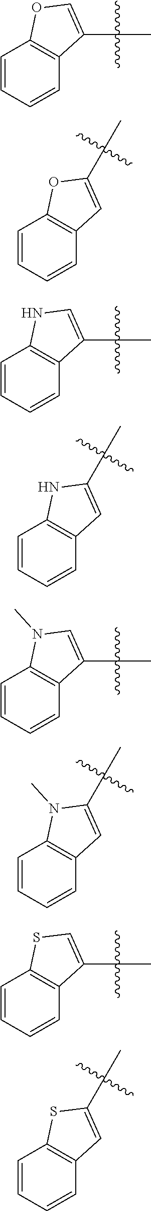

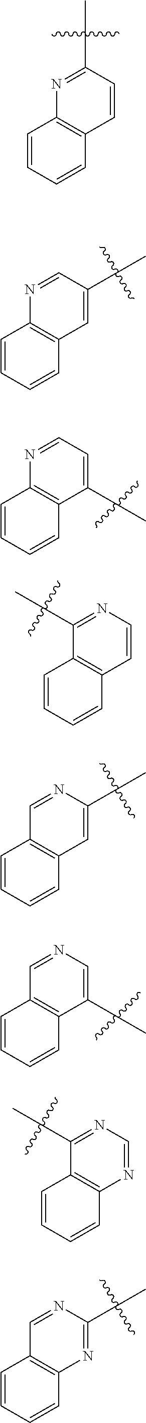

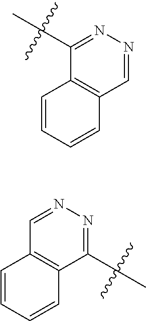 [SMILES: *c1nncc2ccccc12 ; *c1nncc2ccccc12]

As used herein, a “binder” is an excipient that imparts a pharmaceutical composition with enhanced cohesion or tensile strength (e.g., hardness).

As used herein, the term “carrier” means a non-toxic solvent, dispersant, excipient, adjuvant or other material which is mixed with the active ingredient in order to permit the formation of a pharmaceutical composition, i.e., a dosage form capable of administration to the patient.

As used herein, the term “composition” generally refers to a composition of two or more components, usually one or more drugs (e.g., volixibat) and one or more pharmaceutical excipients.

As used herein, the term, “compound” means all stereoisomers, tautomers, and isotopes of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system.

As used herein, the term “cyano” means —CN.

As used herein, the term “cycle” means an optionally substituted ring that is a 6-20 membered aryl, a 3-20 membered heteroaryl having 1-5 heteroatoms independently selected from N, O, and S; a 3-20 membered saturated or partially unsaturated cycloalkyl, or a 3-20 membered saturated or partially unsaturated heterocycloalkyl having 1-5 heteroatoms independently selected from N, O, and S.

As used herein, the term “cycloalkyl” means non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to 15, from 3 to 10, from 3 to 8, from 3 to 6, from 4 to 6, from 3 to 5, or 5 or 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcaranyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, a “disintegrant” is an excipient that hydrates a pharmaceutical composition and aids in tablet dispersion.

As used herein, a “diluent” or “filler” is an excipient that adds bulkiness to a pharmaceutical composition.

As used herein, “DS” refers to drug substance.

As used herein, an “excipient” includes functional and non-functional ingredients in a pharmaceutical composition.

As used herein, a “glidant” is an excipient that imparts a pharmaceutical compositions with enhanced flow properties.

As used herein, the term “halo” means halogen groups including, but not limited to fluoro, chloro, bromo, and iodo.

As used herein, the term “haloalkoxy” means an —O-haloalkyl group. An example of an haloalkoxy group is OCF₃.

As used herein, the term “haloalkyl” means a C₁₋₆ alkyl group having one or more halogen substituents. Examples of haloalkyl groups include, but are not limited to, CF₃, C₂F5, CH₂F, CHF₂, CCl₃, CHCl₂, C₂Cl₅, CH₂CF₃, and the like.

As used herein, the term “heteroatom” or “hetero-atom” refers to an atom that is not carbon atom or an hydrogen atom. In some embodiments, the “heteroatom” or “hetero-atom” is a nitrogen, oxygen, phosphorus, or sulfur atom.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, pyranyl, oxadiazolyl, isoxazolyl, triazolyl, thianthrenyl, pyrazolyl, indolizinyl, isoindolyl, isobenzofuranyl, benzoxazolyl, xanthenyl, 2H-pyrrolyl, pyrrolyl, 3H-indolyl, 4H-quinolizinyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furanyl, phenoxazinyl groups, and the like. Suitable heteroaryl groups include 1,2,3-triazole, 1,2,4-triazole, 5-amino-1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

As used herein, the term “heteroarylalkyl” means a C₁₋₆ alkyl group substituted by a heteroaryl group.

As used herein, the term “heterocycle” or “heterocyclic ring” means a 5- to 7-membered mono- or bicyclic or 7- to 10-membered bicyclic heterocyclic ring system any ring of which may be saturated or unsaturated, and which consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Particularly useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Hetercycloalkyl groups can be mono or polycyclic (e.g., fused, bridged, or spiro systems). In some embodiments, the heterocycloalkyl group has from 1 to 20 carbon atoms, or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthimidyl, and benzo derivatives of heterocycles such as indolene, isoindoline, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “hydroxy” or “hydroxyl” means an —OH group.

As used herein, the term “hydroxyalkyl” or “hydroxylalkyl” means an alkyl group substituted by a hydroxyl group. Examples of a hydroxylalkyl include, but are not limited to, —CH₂OH and —CH₂CH₂OH.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the term “inhibitor” means a substance that reduces measurable amount the activity of an enzyme or receptor. The term “inhibit” means reducing measurable amount the activity of an enzyme or receptor.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent groups, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, a “lubricant” is an excipient that is added to pharmaceutical compositions that are pressed into tablets. The lubricant aids in compaction of granules into tablets and ejection of a tablet of a pharmaceutical composition from a die press.

As used herein, the term “percent” or “%” means the weight percentage of the total weight of the composition (i.e. by weight of the total composition).

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophosphate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. The present embodiments include pharmaceutically acceptable salt of the compounds described herein. The present embodiments also include quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the term “prodrug” means a derivative of a known direct acting drug, which derivative has enhanced delivery characteristics and therapeutic value as compared to the drug, and is transformed into the active drug by an enzymatic or chemical process.

As used herein, the term “purified” means that when isolated, the isolate contains at least 90%, at least 95%, at least 98%, or at least 99% of a compound described herein by weight of the isolate.

As used herein, the term “solid dosage form” generally refers to a pharmaceutical composition, which when used in an oral mode of administration include capsules, tablets, pills, powders and granules. In such solid dosage forms, the active compound is mixed with at least one inert, pharmaceutically acceptable excipient or carrier.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, a “surfactant” is an excipient that imparts pharmaceutical compositions with enhanced solubility and/or wettability.

As used herein, “suitable substituent”, “substituent”, “optional substituent”, or the substituent for any optionally substituted group means a group that does not nullify the synthetic or pharmaceutical utility of the compounds described herein or the intermediates useful for preparing them. Examples of “suitable substituent”, “substituent”, “optional substituent”, or the substituent for any optionally substituted group includes, but are not limited to: C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(phenyl), —N(phenyl)₂, —CHO, —CO(C₁-C₆ alkyl), —CO(phenyl), —CO₂(C₁-C₆ alkyl), and —CO₂(phenyl). In some embodiments, one “suitable substituent”, “substituent”, “optional substituent”, or the substituent for any optionally substituted group is further substituted by one or more groups selected from C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈ alkyl), —N(C₁-C₈ alkyl)₂, —NH(phenyl), —N(phenyl)₂, —CHO, —CO(C₁-C₆ alkyl), —CO(phenyl), —CO₂(C₁-C₆ alkyl), or —CO₂(phenyl). One of skill in art can readily choose a suitable substituent based on the stability and pharmacological and synthetic activity of the compounds described herein.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, elimination or amelioration of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,”, “treatment”, or “treating” mean both therapeutic treatment wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment.

At various places in the present specification, substituents of compounds may be disclosed in groups or in ranges. It is specifically intended that embodiments include each and every individual subcombination of the members of such groups and ranges. For example, the term “C₁₋₆ alkyl” is specifically intended to individually disclose methyl, ethyl, propyl, C₄ alkyl, C₅ alkyl, and C₆ alkyl.

For compounds in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form, for example,

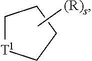

then it is understood that substituent R can occur s number of times on the ring, and R can be a different moiety at each occurrence. Further, in the above example, where the variable T¹ is defined to include hydrogens, such as when T¹ is CH₂, NH, etc., any H can be replaced with a substituent.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It is understood that the present embodiments encompass the use, where applicable, of stereoisomers, diastereomers and optical stereoisomers of the compounds, as well as mixtures thereof. Additionally, it is understood that stereoisomers, diastereomers, and optical stereoisomers of the compounds, and mixtures thereof, are within the scope of the embodiments. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the embodiments unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present embodiments. Cis and trans geometric isomers of the compounds are also included within the scope of the embodiments and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, chiral HPLC, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Resolution Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound of the embodiments. Substantial separation can include compositions containing at least about 50%, at least about 60%, at least about 70%, at least about 80%, at least about 90%, at least about 95%, at least about 97%, or at least about 99% by weight of the compound of the embodiments, or salt thereof. Methods for isolating compounds and their salts are routine in the art.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds also include derivatives referred to as prodrugs.

Embodiments of various compounds and salts thereof are provided. Where a variable is not specifically recited, the variable can be any option described herein, except as otherwise noted or dictated by context.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Provided herein are embodiments directed to a pharmaceutical composition comprising a compound of Formula (I),

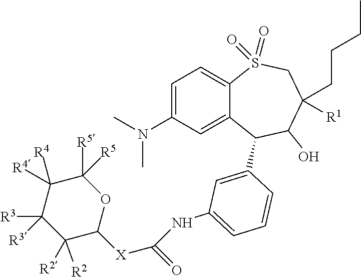

wherein:
X is NH;
R¹ is (C₁-C₆)-alkyl;
R² is OH;
R^{2′} is H;
R³, R^{3′}, R⁴, R^{4′}, R⁵, R^{5′}, are, independently H, Cl, Br, I, OH, (CH₂) OH, CF₃, NO₂, N₃, CN, S(O)ₚ—R⁶, O—S(O)ₚ—R⁶, (C₁-C₆)-alkylene-S(O)ₚ—R⁶, (C₁-C₆)-alkylene-O—S(O)ₚ—R⁶, COOH, COO(C₁-C₆)alkyl, CONH₂, CONH(C₁-C₆)alkyl, CON[(C₁-C₆)alkyl]₂, (C₁-C₆)-alkyl, (C₂-C₆)alkenyl, (C₂-C₆)-alkynyl, O—(C₁-C₆)-alkyl, where one, more than one, or all hydrogen(s) in the alkyl radicals may be replaced by fluorine, phenyl, —(CH₂)-phenyl, —(CH₂)₂-phenyl, O-phenyl, O—(CH₂)ₘ-phenyl, —(CH₂)—O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂;
wherein at least one of the radicals R³, R^{3′}, R⁴, R^{4′}, R⁵, and R^{5′} has the meaning of (C₁-C₆)-alkylene-O—S(O)ₚ—R⁶ and another has the meaning of —O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂;
R⁶ is H, OH, (C₁-C₆)-alkyl, NH₂, NH(C₁-C₆)-alkyl or N((C₁-C₆)-alkyl)₂;
n is 2, 3, 4, 5 or 6;
m is 1, 2, 3, 4, 5 or 6;
p is 0, 1 or 2;
or a pharmaceutically acceptable salt thereof, and one or more excipients selected from the group consisting of:
   (i) a diluent or a filler, and
   (ii) a disintegrant.

Generally, the compound of Formula (I) are administered in a therapeutically effective amount. The amount of the compound actually administered will typically be determined by a physician, in the light of the relevant circumstances, including the condition to be treated, the chosen route of administration, the actual compound—administered, the age, weight, and response of the individual patient, the severity of the patient's symptoms, and the like.

The pharmaceutical compositions of the compound of Formula (I) can be administered by a variety of routes including oral, rectal, intraocular, transdermal, subcutaneous, intravenous, intramuscular, intraperitoneal, intradermal, directly into cerebrospinal fluid, intratracheal, and intranasal. Depending on the intended route of delivery, the compound of Formula (I) is preferably formulated as oral compositions.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the active compound is usually a minor component with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Pharmaceutical compositions containing the compound of Formula (I) can be prepared in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, soft and hard gelatin capsules, suppositories, sterile injectable solutions, and sterile packaged powders.

In some embodiments, the composition comprising a compound of Formula (I) further comprises a channeling agent.

In some embodiments, the composition comprising a compound of Formula (I) further comprises a glidant, a lubricant, or a combination thereof.

In some embodiments, the composition comprises a compound of Formula (I), or a pharmaceutically acceptable salt thereof, and one or more selected from the group consisting of:
(i) a diluent or a filler,
(ii) a disintegrant,
(iii) a channeling agent,
(iv) a glidant, and
(v) a lubricant.

In some embodiments, the composition comprises a compound of Formula (I), or a pharmaceutically acceptable salt thereof, and
(i) a diluent or a filler,
(ii) a disintegrant,
(iii) a channeling agent,
(iv) a glidant, and
(v) a lubricant.

In some embodiments, the composition further comprises comprising a binder.

In some embodiments, the composition of Formula (I) has R^{5′} that is (C₁-C₆)-alkylene-S(O)₂—R⁶.

In some embodiments, the compound of Formula (I) has the structure selected from the group consisting of Formula (II), (III), or (IV), or a pharmaceutically acceptable salt thereof:

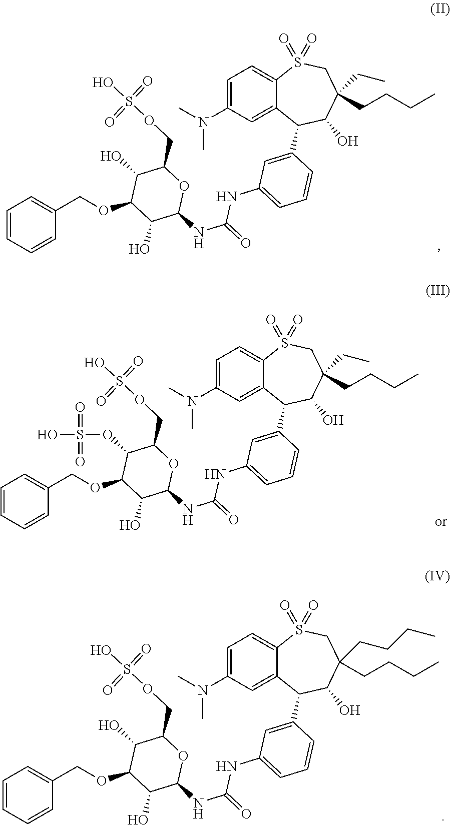 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O ; CCCC[C@]1(CC)CS(=O)(=O)c2ccc(N(C)C)cc2[C@@H](c2cccc(NC(=O)N[C@@H]3O[C@H](COS(=O)(=O)O)[C@@H](OS(=O)(=O)O)[C@H](OCc4ccccc4)[C@H]3O)c2)[C@H]1O ; CCCCC1(CCCC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O]

In some embodiments, the compound of Formula (I) has the structure selected from the group consisting of Formula (II) or (III), or a pharmaceutically acceptable salt thereof:

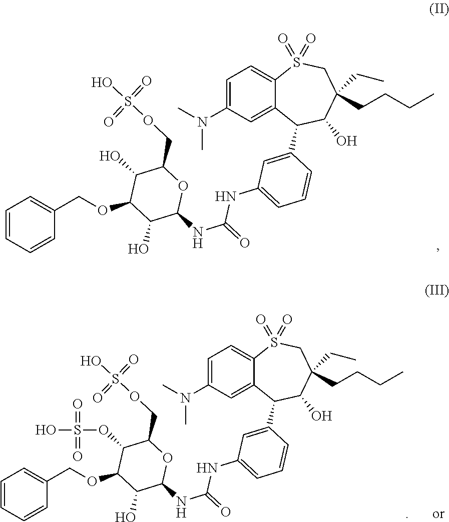 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O ; CCCC[C@]1(CC)CS(=O)(=O)c2ccc(N(C)C)cc2[C@@H](c2cccc(NC(=O)N[C@@H]3O[C@H](COS(=O)(=O)O)[C@@H](OS(=O)(=O)O)[C@H](OCc4ccccc4)[C@H]3O)c2)[C@H]1O]

In some embodiments, the compound of Formula (I) has the structure of Formula (II), or a pharmaceutically acceptable salt thereof:

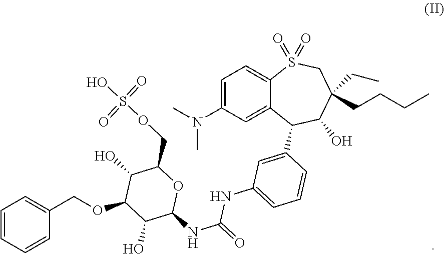 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O]

In some embodiments, the composition comprises the pharmaceutically acceptable salt of the compound of Formula (I), wherein the pharmaceutically acceptable salt is an ammonium salt.

In some embodiments, the composition comprises the pharmaceutically acceptable salt of the compound of Formula (I), wherein the pharmaceutically acceptable salt is an alkali metal or alkaline earth metal salt.

In some embodiments, the composition comprises the pharmaceutically acceptable salt of the compound of Formula (I), wherein the pharmaceutically acceptable salt is a potassium salt.

In some embodiments, the composition comprises the compound of Formula (II) as a potassium ethanolate hydrate:

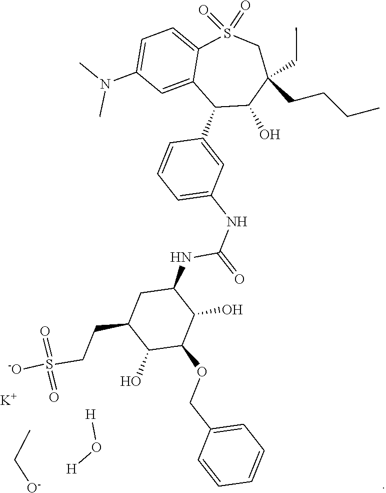

In some embodiments, the composition comprises the pharmaceutically acceptable salt of the compound of Formula (I), wherein the pharmaceutically acceptable salt is a zinc salt.

In some embodiments, the composition comprises the compound of Formula (I) having the structure of Formula (II), or a pharmaceutically acceptable salt thereof, and
(i) a diluent or a filler,
(ii) a disintegrant,
(iii) optionally a channeling agent,
(iv) a glidant, and
(v) a lubricant.

Provided herein are embodiments directed to a pharmaceutical composition comprising the compound of Formula (I), or a pharmaceutically acceptable salt thereof, that is less than about 70%, 65%, 60%, 55%, 50%, 45%, 40%, 35%, 30%, 25%, 20%, 15%, 12.5%, 10%, 7.5%, 5%, 3%, 2%, or 1% by weight of the total composition. In some embodiments, the compound of Formula (I), or a pharmaceutically acceptable salt thereof, is about 70%, 65%, 60%, 55%, 50%, 45%, 40%, 35%, 30%, 25%, 20%, 15%, 12.5%, 10%, 7.5%, 5%, 3%, 2%, or 1% by weight of the total composition. In some embodiments, the compound of Formula (I), or a pharmaceutically acceptable salt thereof, is about 1%-30%, 3%-30%, 5%-30%, 7.5%-30%, 10%-30%, 15%-30%, 20%-30%, 1%-25%, 3%-25%, 5%-25%, 7.5%-25%, 10%-25%, 15%-25%, 20%-25%, 1%-20%, 3%-20%, 5%-20%, 7.5%-20%, 10%-20%, 15%-20%, 1%-15%, 3%-15%, 5%-15%, 7.5%-15%, 10%-15%, 1%-12.5%, 3%-12.5%, 5%-12.5%, 7.5%-12.5%, 10%-12.5%, 1%-10%, 3%-10%, 5%-10%, or 7.5%-10% by weight of the total composition.

In some embodiments, the compound of Formula (I), or a pharmaceutically acceptable salt thereof, is less than about 60% by weight of the total composition.

In some embodiments, the compound of Formula (I), or a pharmaceutically acceptable salt thereof, is less than about 50% by weight of the total composition.

In some embodiments, the compound of Formula (I), or a pharmaceutically acceptable salt thereof, is about 5% to about 20% by weight of the total composition.

In some embodiments, the compound of Formula (I), or a pharmaceutically acceptable salt thereof, is about 7.5% to about 15%, about 2% to about 10%, about 3% to about 10%, or about 10% to about 20% by weight of the total composition.

In some embodiments, the compound of Formula (I), or a pharmaceutically acceptable salt thereof, is about 7.5% by weight of the total composition.

In some embodiments, the compound of Formula (I), or a pharmaceutically acceptable salt thereof, is about 15% by weight of the total composition.

Provided herein are embodiments directed to a pharmaceutical composition comprising the compound of Formula (I), or a pharmaceutically acceptable salt thereof, that is in an amount of less than about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, 5 mg, 3 mg, 2 mg, or 1 mg. In some embodiments, the compound of Formula (I), or a pharmaceutically acceptable salt thereof, is in an amount of about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 12.5 mg, 10 mg, 7.5 mg, 5 mg, 3 mg, 2 mg, or 1 mg. In some embodiments, the compound of Formula (I), or a pharmaceutically acceptable salt thereof, is in an amount of about 1 mg-150 mg, 3 mg-150 mg, 5 mg-150 mg, 7.5 mg-150 mg, 10 mg-150 mg, 12.5 mg-150 mg, 15 mg-150 mg, 20 mg-150 mg, 25 mg-150 mg, 30 mg-150 mg, 35 mg-150 mg, 40 mg-150 mg, 1 mg-120 mg, 3 mg-120 mg, 5 mg-120 mg, 7.5 mg-120 mg, 10 mg-120 mg, 12.5 mg-120 mg, 15 mg-120 mg, 20 mg-120 mg, 25 mg-120 mg, 30 mg-120 mg, 35 mg-120 mg, 40 mg-120 mg, 1 mg-100 mg, 3 mg-100 mg, 5 mg-100 mg, 7.5 mg-100 mg, 10 mg-100 mg, 12.5 mg-100 mg, 15 mg-100 mg, 20 mg-100 mg, 25 mg-100 mg, 30 mg-100 mg, 35 mg-100 mg, 40 mg-100 mg, 1 mg-80 mg, 3 mg-80 mg, 5 mg-80 mg, 7.5 mg-80 mg, 10 mg-80 mg, 12.5 mg-80 mg, 15 mg-80 mg, 20 mg-80 mg, 25 mg-80 mg, 30 mg-80 mg, 35 mg-80 mg, 40 mg-80 mg, 1 mg-60 mg, 3 mg-60 mg, 5 mg-60 mg, 7.5 mg-60 mg, 10 mg 60 mg, 12.5 mg-60 mg, 15 mg-60 mg, 20 mg-60 mg, 25 mg-60 mg, 30 mg-60 mg, 35 mg-60 mg, 40 mg-60 mg, 1 mg-50 mg, 3 mg-50 mg, 5 mg-50 mg, 7.5 mg-50 mg, 10 mg-50 mg, 12.5 mg-50 mg, 15 mg-50 mg, 20 mg-50 mg, 25 mg-50 mg, 30 mg-50 mg, 35 mg-50 mg, 40 mg-50 mg, 1 mg-40 mg, 3 mg-40 mg, 5 mg-40 mg, 7.5 mg-40 mg, 10 mg-40 mg, 12.5 mg-40 mg, 15 mg-40 mg, 20 mg-40 mg, 25 mg-40 mg, 30 mg-40 mg, 35 mg-40 mg, 1 mg-30 mg, 3 mg-30 mg, 5 mg-30 mg, 7.5 mg-30 mg, 10 mg-30 mg, 12.5 mg-30 mg, 15 mg-30 mg, 20 mg-30 mg, 25 mg-30 mg, 1 mg-20 mg, 3 mg-20 mg, 5 mg-20 mg, 7.5 mg-20 mg, 10 mg-20 mg, 12.5 mg-20 mg, 15 mg-20 mg, 1 mg-15 mg, 3 mg-15 mg, 5 mg-15 mg, 7.5 mg-15 mg, 10 mg-15 mg, 12.5 mg-15 mg, 1 mg-10 mg, 3 mg-10 mg, 5 mg-10 mg, 7.5 mg-10 mg, 1 mg-7.5 mg, 3 mg-7.5 mg, 5 mg-7.5 mg, 1 mg-5 mg, or 3 mg-5 mg.

In some embodiments, the compound of Formula (I), or a pharmaceutically acceptable salt thereof, is in an amount of less than about 100 mg.

In some embodiments, the compound of Formula (I), or a pharmaceutically acceptable salt thereof, is in an amount of less than about 50 mg.

In some embodiments, the compound of Formula (I), or a pharmaceutically acceptable salt thereof, is in an amount of about 5 mg to about 20 mg.

In some embodiments, the compound of Formula (I), or a pharmaceutically acceptable salt thereof, is in an amount of about 5 mg.

In some embodiments, the compound of Formula (I), or a pharmaceutically acceptable salt thereof, is in an amount of about 20 mg.

In some embodiments, the compound of Formula (I), or a pharmaceutically acceptable salt thereof, is in an amount of less than about 5 mg, about 5 mg to 20 mg, or more than about 20 mg.

In some embodiments, the compound of Formula (I), or a pharmaceutically acceptable salt thereof, is in an amount of less than about 10 mg, about 10 mg to 40 mg, or more than about 40 mg.

In some embodiments, the compound of Formula (I), or a pharmaceutically acceptable salt thereof, is in an amount of about 10 mg to about 40 mg.

In some embodiments, the compound of Formula (I), or a pharmaceutically acceptable salt thereof, in an amount of about 10 mg.

In some embodiments, the compound of Formula (I), or a pharmaceutically acceptable salt thereof, is in an amount of about 40 mg.

In some embodiments, the compound of Formula (I), or a pharmaceutically acceptable salt thereof, is in an amount of about 80 mg.

In some embodiments, wherein the diluent or the filler is selected from the group consisting of a sugar, dextrates, dextrin, dextrose, lactose, mannitol, sorbitol, starch, cellulose, and modified celluloses, or a combination thereof.

In some embodiments, the diluent or the filler is microcrystalline cellulose (MCC), lactose, mannitol, or a combination thereof.

In some embodiments, the diluent or the filler is a mixture of MCC and lactose, a mixture of MCC and mannitol, or a mixture of lactose and mannitol.

In some embodiments, the diluent or the filler is less than about 99%, 97%, 95%, 93%, 90%, 86%, 85%, 80%, 75%, 73%, 71%, 70%, 65%, 60%, 59%, 55%, 50%, 45%, 40%, 35%, or 30% by weight of the total composition. In some embodiments, the diluent or the filler is about 99%, 97%, 95%, 93%, 90%, 86%, 85%, 80%, 75%, 73%, 71%, 70%, 65%, 60%, 59%, 55%, 50%, 45%, 40%, 35%, or 30% by weight of the total composition. In some embodiments, the diluent or the filler is about 30%-95%, 40%-95%, 50%-95%, 60%-95%, 65%-95%, 70%-95%, 80%-95%, 90%-95%, 30%-90%, 40%-90%, 50%-90%, 60%-90%, 65%-90%, 70%-90%, 80%-90%, 30%-80%, 40%-80%, 50%-80%, 60%-80%, 65%-80%, 70%-80%, 30%-85%, 40%-85%, 50%-85%, 60%-85%, 65%-85%, 70%-85%, 30%-75%, 40%-75%, 50%-75%, 60%-75%, 65%-75%, 70%-75%, 30%-70%, 40%-70%, 50%-70%, 65%-70%, 30%-65%, 40%-65%, 50%-65%, 60%-65%, or 60%-70%.

In some embodiments, the combined amount of the diluent or the filler and the channeling agent is less than about 99%, 97%, 95%, 93%, 90%, 86%, 85%, 80%, 75%, 73%, 71%, 70%, 65%, 60%, 59%, 55%, 50%, 45%, 40%, 35%, or 30% by weight of the total composition. In some embodiments, the combined amount of the diluent or the filler and the channeling agent is about 99%, 97%, 95%, 93%, 90%, 86%, 85%, 80%, 75%, 73%, 71%, 70%, 65%, 60%, 59%, 55%, 50%, 45%, 40%, 35%, or 30% by weight of the total composition. In some embodiments, the combined amount of the diluent or the filler and the channeling agent is about 30%-95%, 40%-95%, 50%-95%, 60%-95%, 65%-95%, 70%-95%, 80%-95%, 90%-95%, 30%-90%, 40%-90%, 50%-90%, 60%-90%, 65%-90%, 70%-90%, 80%-90%, 30%-80%, 40%-80%, 50%-80%, 60%-80%, 65%-80%, 70%-80%, 30%-85%, 40%-85%, 50%-85%, 60%-85%, 65%-85%, 70%-85%, 30%-75%, 40%-75%, 50%-75%, 60%-75%, 65%-75%, 70%-75%, 30%-70%, 40%-70%, 50%-70%, 65%-70%, 30%-65%, 40%-65%, 50%-65%, 60%-65%, or 60%-70%.

In some embodiments, the diluent or the filler is less than about 97% by weight of the total composition.

In some embodiments, the diluent or the filler is in an amount of about 55% to about 90% by weight of the total composition.

In some embodiments, the diluent or the filler is about 59%, about 71%, about 73%, or about 86% by weight of the total composition.

In some embodiments, the diluent or the filler comprises MCC and/or lactose. In some embodiments, the diluent or the filler that is MCC or lactose is in an amount of about 70%, 65%, 60%, 58%, 56%, 50%, 46%, 48%, 44%, 40%, 30%, 25%, 20%, 15%, 14%, 10%, 5%, 3%, or 1%. In some embodiments, the diluent or the filler that is MCC or lactose is in an amount of about 30%-95%, 40%-95%, 50%-95%, 60%-95%, 65%-95%, 70%-95%, 80%-95%, 90%-95%, 30%-90%, 40%-90%, 50%-90%, 60%-90%, 65%-90%, 70%-90%, 80%-90%, 30%-80%, 40%-80%, 50%-80%, 60%-80%, 65%-80%, 70%-80%, 30%-85%, 40%-85%, 50%-85%, 60%-85%, 65%-85%, 70%-85%, 30%-75%, 40%-75%, 50%-75%, 60%-75%, 65%-75%, 70%-75%, 30%-70%, 40%-70%, 50%-70%, 65%-70%, 30%-65%, 40%-65%, 50%-65%, 60%-65%, or 60%-70%. In some embodiments, the diluent or the filler that is MCC or lactose is in an amount of about 5%-70%, 10%-70%, 15%-70%, 20%-70%, 40%-70%, 50%-70%, 5%-60%, 10%-60%, 15%-60%, 20%-60%, 40%-60%, 50%-60%, 5%-50%, 10%-50%, 15%-50%, 20%-50%, 40%-50%, 5%-40%, 10%-40%, 15%-40%, 20%-40%, 5%-30%, 10%-30%, 15%-30%, 20%-30%, 5%-20%, 10%-20%, or 15%-20%.

In some embodiments, the diluent or the filler comprises MCC in an amount of about 40% to about 60% by weight of the total composition.

In some embodiments, the diluent or the filler comprises MCC in an amount of about 44%, about 46%, about 48%, about 56%, or about 58% by weight of the total composition.

In some embodiments, the diluent or the filler comprises lactose in an amount of about 10% to about 40% by weight of the total composition.

In some embodiments, the diluent or the filler comprises lactose in an amount of about 14%, about 15%, about 25%, or about 40% by weight of the total composition.

In some embodiments, the diluent or the filler comprises mannitol in an amount of about 10% to about 20% by weight of the total composition.

In some embodiments, the diluent or the filler comprises mannitol in an amount of about 14% by weight of the total composition.

In some embodiments, the channeling agent is selected from the group consisting of sodium chloride and polyethylene glycol (PEG), or a combination thereof.

In some embodiments, the channeling agent is sodium chloride, sugar, citric acid, sodium citrate, sodium bicarbonate, potassium chloride, potassium citrate, dextrins, fructose, saccharin sodium, or xylitol.

In some embodiments, the channeling agent is PEG.

In some embodiments, the channeling agent is PEG 1500.

In some embodiments, the channeling agent is in an amount of less than about 30%, 25%, 20%, 15%, 10%, 5%, 3%, or 1% by weight of the total composition. In some embodiments, the channeling agent is in an amount of about 30%, 25%, 20%, 15%, 14%, 10%, 5%, 3%, or 1% by weight of the total composition. In some embodiments, the channeling agent is in an amount of about 1%-30%, 5%-30%, 10%-30%, 15%-30%, 20%-30%, 25%-30%, 1%-20%, 5%-20%, 10%-20%, 15%-20%, 1%-15%, 5%-15%, 10%-15%, 1%-10%, 5%-10%, or 1%-5% by weight of the total composition.

In some embodiments, the channeling agent is in an amount of less than about 20% by weight of the total composition.

In some embodiments, the channeling agent is in an amount about 5% to about 15% by weight of the total composition.

In some embodiments, the channeling agent is in an amount of about 5%, about 14%, or about 15% by weight of the total composition.

In some embodiments, the disintegrant is in an amount of less than about 30%, 25%, 20%, 15%, 10%, 5%, 3%, 2%, or 1% by weight of the total composition. In some embodiments, the disintegrant is in an amount of about 30%, 25%, 20%, 15%, 10%, 5%, 3%, 2%, or 1%. In some embodiments, the disintegrant is in an amount of 2%-30%, 5%-30%, 10%-30%, 15%-30%, 20%-30%, 2%-20%, 5%-20%, 10%-20%, 15%-20%, 2%-15%, 5%-15%, 10%-15%, 2%-10%, 5%-10%, or 2%-5% by weight of the total composition.

In some embodiments, the disintegrant is selected from the group consisting of croscarmellose sodium, crospovidone, starch, and sodium starch glycolate, or a combination thereof.

In some embodiments, the disintegrant is in an amount of less than about 30%.

In some embodiments, the disintegrant is in an amount of less than about 20% by weight of the total composition.

In some embodiments, the disintegrant is in an amount of about 2% to about 10% by weight of the total composition.

In some embodiments, the disintegrant is in an amount of about 5% or about 10% by weight of the total composition.

In some embodiments, the glidant is selected from the group consisting of silicon dioxide, magnesium stearate, talc and corn starch, or a combination thereof.

In some embodiments, the glidant comprises silicon dioxide.

In some embodiments, the glidant is in an amount of less about 10%, 5%, 2%, or 1%. In some embodiments, the glidant is in an amount of about 0.1%, 0.3%, 0.5%, 1%, 1.5, 2%, 2.5%, 3%, 3.5%, 4%, 4.5%, 5%, 8%, or 10%. In some embodiments, the glidant is in an amount of about 0.1%-10%, 0.3%-10%, 0.5%-10%, 1%-10%, 2%-10%, 5%-10%0.1%-5%, 0.3% 5%, 0.5%-5%, 1%-5%, 2%-5%, 0.1%-2%, 0.3%-2%, 0.5%-2%, 1%-2%, 0.1%-1%, 0.3%-1%, or 0.5%-1%.

In some embodiments, the glidant is in an amount of less than about 2% by weight of the total composition.

In some embodiments, the glidant is in an amount of about 1% by weight of the total composition.

In some embodiments, the lubricant is selected from the group consisting of magnesium stearate, talc, calcium stearate, zinc stearate, sodium stearate, sodium stearyl fumarate, stearic acid, aluminum stearate, leucine, glyceryl behenate, and hydrogenated vegetable oil, or a combination thereof.

In some embodiments, the lubricant comprises magnesium stearate.

In some embodiments, the lubricant is in an amount of less than 10%, 5%, 2%, or 1%. In some embodiments, the lubricant is in an amount of about 0.5%, 1%, 1.5, 2%, 2.5%, 3%, 3.5%, 4%, 4.5%, 5%, 8%, or 10%. In some embodiments, the lubricant is in an amount of about 0.1%-10%, 0.3%-10%, 0.5%-10%, 1%-10%, 2%-10%, 5%-10%0.1%-5%, 0.3%-5%, 0.5%-5%, 1%-5%, 2%-5%, 0.1%-2%, 0.3%-2%, 0.5%-2%, 1%-2%, 0.1%-1%, 0.3%-1%, or 0.5%-1%.

In some embodiments, the lubricant is in an amount of less than about 2% by weight of the total composition.

In some embodiments, the lubricant is in an amount of about 0.5% or about 1% by weight of the total composition.

In some embodiments, the composition further comprises a binder selected from the group consisting of polyvinylpyrrolidone, dibasic calcium phosphate, sucrose, corn starch, and modified cellulose, or a combination thereof.

Provided herein are embodiments directed to a composition comprising the compound of Formula (I), or a pharmaceutically acceptable salt thereof, in the amount of about 5%, 10%, 15%, 20%, 25%, 30%, 40%, 50%, 60%, 70%, or 80%. In some embodiments, a composition comprises, the compound of Formula (I) in the amount of about 5%-80%, 10%-80%, 15%-80%, 20%-80%, 25%-80%, 30%-80%, 40%-80%, 5%-60%, 10%-60%, 15%-60%, 20%-60%, 25%-60%, 30%-60%, 40%-60%, 5%-40%, 10%-40%, 15%-40%, 20%-40%, 25%-40%, 30%-40%, 5%-20%, 10%-20%, 15%-20%, or 5%-10%.

Also provided herein are embodiments directed to a composition comprising the compound of Formula (II), or a pharmaceutically acceptable salt thereof, in the amount of about 5%, 10%, 15%, 20%, 25%, 30%, 40%, 50%, 60%, 70%, or 80%. In some embodiments, a composition comprises the compound of Formula (II), or a pharmaceutically acceptable salt thereof, in the amount of about 5%-80%, 10%-80%, 15%-80%, 20%-80%, 25%-80%, 30%-80%, 40%-80%, 5%-60%, 10%-60%, 15%-60%, 20%-60%, 25%-60%, 30%-60%, 40%-60%, 5%-40%, 10%-40%, 15%-40%, 20%-40%, 25%-40%, 30%-40%, 5%-20%, 10%-20%, 15%-20%, or 5%-10%.

Also provided herein are embodiments directed to a composition comprising the compound of Formula (III), or a pharmaceutically acceptable salt thereof, in the amount of about 5%, 10%, 15%, 20%, 25%, 30%, 40%, 50%, 60%, 70%, or 80%. In some embodiments, a composition comprises the compound of Formula (III), or a pharmaceutically acceptable salt thereof, in the amount of about 5%-80%, 10%-80%, 15%-80%, 20%-80%, 25%-80%, 30%-80%, 40%-80%, 5%-60%, 10%-60%, 15%-60%, 20%-60%, 25%-60%, 30%-60%, 40%-60%, 5%-40%, 10%-40%, 15%-40%, 20%-40%, 25%-40%, 30%-40%, 5%-20%, 10%-20%, 15%-20%, or 5%-10%.

Also provided herein are embodiments directed to a composition comprising the compound of Formula (IV)), or a pharmaceutically acceptable salt thereof, in the amount of about 5%, 10%, 15%, 20%, 25%, 30%, 40%, 50%, 60%, 70%, or 80%. In some embodiments, a composition comprises the compound of Formula (IV)), or a pharmaceutically acceptable salt thereof, in the amount of about 5%-80%, 10%-80%, 15%-80%, 20%-80%, 25%-80%, 30%-80%, 40%-80%, 5%-60%, 10%-60%, 15%-60%, 20%-60%, 25%-60%, 30%-60%, 40%-60%, 5%-40%, 10%-40%, 15%-40%, 20%-40%, 25%-40%, 30%-40%, 5%-20%, 10%-20%, 15%-20%, or 5%-10%.

In some embodiments, the composition comprises about 5% to about 60% of the compound of Formula (II), or a pharmaceutically acceptable salt thereof, by weight of the total composition, and
(i) a diluent or filler,
(ii) optionally a channeling agent,
(iii) a disintegrant,
(iv) a glidant, and
(v) a lubricant,
wherein when the channeling agent is not present, the amount of the diluent or filler is about 70% to about 90% by weight of the total composition, or when the channeling agent is present, the combined amount of the diluent or filler and the channeling agent is about 70% to about 90% by weight of the total composition.

In some embodiments, the composition comprises about 5% to about 20% of the compound of Formula (II), or a pharmaceutically acceptable salt thereof, by weight of the total composition, and
(i) a diluent or filler,
(ii) optionally a channeling agent,
(iii) a disintegrant,
(iv) a glidant, and
(v) a lubricant,
wherein when the channeling agent is not present, the amount of the diluent or filler is about 70% to about 90% by weight of the total composition, or when the channeling agent is present, the combined amount of the diluent or filler and the channeling agent is about 70% to about 90% by weight of the total composition.

In some embodiments, the composition comprises about 5% to about 10% of the compound of Formula (II), or a pharmaceutically acceptable salt thereof, by weight of the total composition, and
(i) a diluent or filler,
(ii) optionally a channeling agent,
(iii) a disintegrant,
(iv) a glidant, and
(v) a lubricant,
wherein when the channeling agent is not present, the amount of the diluent or filler is about 70% to about 90% by weight of the total composition, or when the channeling agent is present, the combined amount of the diluent or filler and the channeling agent is about 70% to about 90% by weight of the total composition.

In some embodiments, the composition comprises about 5% to about 20% the compound of Formula (II), or a pharmaceutically acceptable salt thereof, by weight of the total composition, and
(i) a diluent or filler in an amount of about 65% to about 85% by weight of the total composition,
(ii) a channeling agent in an amount of about 10% to about 20% by weight of the total composition,
(iii) a disintegrant,
(iv) a glidant, and
(v) a lubricant.

In some embodiments, the composition comprises about 5% to about 10% the compound of Formula (II), or a pharmaceutically acceptable salt thereof, by weight of the total composition, and
(i) a diluent or filler in an amount of about 65% to about 75% by weight of the total composition,
(ii) a channeling agent in an amount of about 10% to about 20% by weight of the total composition,
(iii) a disintegrant,
(iv) a glidant, and
(v) a lubricant.

In some embodiments, the composition comprises:
a disintegrant in an amount of about 5% to about 10% by weight of the total composition,
a glidant in an amount of about 0.5% to about 2% by weight of the total composition, and
a lubricant in an amount of about 0.5% to about 2% by weight of the total composition.

In some embodiments, the composition comprises about 15% the compound of Formula (II), or a pharmaceutically acceptable salt thereof, and
(i) about 44% MCC,
(ii) about 14% lactose,
(iii) about 14% sodium chloride,
(iv) about 10% sodium starch glycolate,
(v) about 1% silicon dioxide, and
(vi) about 1% magnesium stearate,
wherein each percentage is a weight percentage by weight of the total composition.

In some embodiments, the composition comprises about 15% the compound of Formula (II), or a pharmaceutically acceptable salt thereof, and
(i) about 44% MCC,
(ii) about 14% mannitol,
(iii) about 14% sodium chloride,
(iv) about 10% sodium starch glycolate,
(v) about 1% silicon dioxide, and
(vi) about 1% magnesium stearate,
wherein each percentage is a weight percentage by weight of the total composition.

In some embodiments, the composition comprises about 15% the compound of Formula (II), or a pharmaceutically acceptable salt thereof, and
(i) about 48% MCC,
(ii) about 25% lactose,
(iii) about 5% sodium chloride,
(iv) about 5% sodium starch glycolate,
(v) about 1% silicon dioxide, and
(vi) about 1% magnesium stearate,
wherein each percentage is a weight percentage by weight of the total composition.

In some embodiments, the composition comprises about 15% the compound of Formula (II), or a pharmaceutically acceptable salt thereof, and
(i) about 58% MCC,
(ii) about 15% lactose,
(iii) about 5% sodium chloride,
(iv) about 5% sodium starch glycolate,
(v) about 1% silicon dioxide, and
(vi) about 1% magnesium stearate,
wherein each percentage is a weight percentage by weight of the total composition.

In some embodiments, the composition comprises about 7.5% the compound of Formula (II), or a pharmaceutically acceptable salt thereof, and
(i) about 56% MCC,
(ii) about 15% lactose,
(iii) about 15% sodium chloride,
(iv) about 5% sodium starch glycolate,
(v) about 1% silicon dioxide, and
(vi) about 0.5% magnesium stearate,
wherein each percentage is a weight percentage by weight of the total composition.

In some embodiments, the composition comprises about 7.5% the compound of Formula (II), or a pharmaceutically acceptable salt thereof, and
(i) about 46% MCC,
(ii) about 40% lactose,
(iii) about 5% sodium starch glycolate,
(iv) about 1% silicon dioxide, and
(v) about 0.5% magnesium stearate,
wherein each percentage is a weight percentage by weight of the total composition.

In some embodiments, the composition comprises about 6.5% the compound of Formula (II), or a pharmaceutically acceptable salt thereof, and
(i) about 56% MCC,
(ii) about 15% lactose,
(iii) about 15% sodium chloride,
(iv) about 5% sodium starch glycolate,
(v) about 1% silicon dioxide, and
(vi) about 0.5% magnesium stearate,
wherein each percentage is a weight percentage by weight of the total composition.

Provided herein are embodiments directed to a pharmaceutical composition comprising the compound of Formula (II), (III), or (IV), or a pharmaceutically acceptable salt thereof, that is in an amount of less than about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, 5 mg, 3 mg, 2 mg, or 1 mg. In some embodiments, the compound of Formula (I), or a pharmaceutically acceptable salt thereof, is in an amount of about 200 mg, 180 mg, 150 mg, 120 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 12.5 mg, 10 mg, 7.5 mg, 5 mg, 3 mg, 2 mg, or 1 mg. In some embodiments, the compound of Formula (II), (III), or (IV), or a pharmaceutically acceptable salt thereof, is in an amount of about 1 mg-150 mg, 3 mg-150 mg, 5 mg-150 mg, 7.5 mg-150 mg, 10 mg-150 mg, 12.5 mg-150 mg, 15 mg-150 mg, 20 mg-150 mg, 25 mg-150 mg, 30 mg-150 mg, 35 mg-150 mg, 40 mg-150 mg, 1 mg-120 mg, 3 mg-120 mg, 5 mg-120 mg, 7.5 mg-120 mg, 10 mg-120 mg, 12.5 mg-120 mg, 15 mg-120 mg, 20 mg-120 mg, 25 mg-120 mg, 30 mg-120 mg, 35 mg-120 mg, 40 mg-120 mg, 1 mg-100 mg, 3 mg-100 mg, 5 mg-100 mg, 7.5 mg-100 mg, 10 mg-100 mg, 12.5 mg-100 mg, 15 mg-100 mg, 20 mg-100 mg, 25 mg-100 mg, 30 mg-100 mg, 35 mg-100 mg, 40 mg-100 mg, 1 mg-80 mg, 3 mg-80 mg, 5 mg-80 mg, 7.5 mg-80 mg, 10 mg-80 mg, 12.5 mg-80 mg, 15 mg-80 mg, 20 mg-80 mg, 25 mg-80 mg, 30 mg-80 mg, 35 mg-80 mg, 40 mg-80 mg, 1 mg-60 mg, 3 mg-60 mg, 5 mg-60 mg, 7.5 mg-60 mg, 10 mg-60 mg, 12.5 mg-60 mg, 15 mg-60 mg, 20 mg-60 mg, 25 mg-60 mg, 30 mg-60 mg, 35 mg-60 mg, 40 mg-60 mg, 1 mg-50 mg, 3 mg-50 mg, 5 mg-50 mg, 7.5 mg-50 mg, 10 mg-50 mg, 12.5 mg-50 mg, 15 mg-50 mg, 20 mg-50 mg, 25 mg-50 mg, 30 mg-50 mg, 35 mg-50 mg, 40 mg-50 mg, 1 mg-40 mg, 3 mg-40 mg, 5 mg-40 mg, 7.5 mg-40 mg, 10 mg-40 mg, 12.5 mg-40 mg, 15 mg-40 mg, 20 mg-40 mg, 25 mg-40 mg, 30 mg-40 mg, 35 mg-40 mg, 1 mg-30 mg, 3 mg-30 mg, 5 mg-30 mg, 7.5 mg-30 mg, 10 mg-30 mg, 12.5 mg-30 mg, 15 mg-30 mg, 20 mg-30 mg, 25 mg-30 mg, 1 mg-20 mg, 3 mg-20 mg, 5 mg-20 mg, 7.5 mg-20 mg, 10 mg-20 mg, 12.5 mg-20 mg, 15 mg-20 mg, 1 mg-15 mg, 3 mg-15 mg, 5 mg-15 mg, 7.5 mg-15 mg, 10 mg-15 mg, 12.5 mg-15 mg, 1 mg-10 mg, 3 mg-10 mg, 5 mg-10 mg, 7.5 mg-10 mg, 1 mg-7.5 mg, 3 mg-7.5 mg, 5 mg-7.5 mg, 1 mg-5 mg, or 3 mg-5 mg.

In some embodiments, the compound of Formula (II), or a pharmaceutically acceptable salt thereof, is in amount of less than about 100 mg.

In some embodiments, the compound of Formula (II), or a pharmaceutically acceptable salt thereof, is in amount of less than about 50 mg.

In some embodiments, the compound of Formula (II), or a pharmaceutically acceptable salt thereof, is in an amount of about 5 mg to about 20 mg.

In some embodiments, the compound of Formula (II), or a pharmaceutically acceptable salt thereof, is in an amount of less than about 5 mg, about 5 mg to 20 mg, or more than about 20 mg.

In some embodiments, the compound of Formula (II), or a pharmaceutically acceptable salt thereof, is in an amount of less than about 10 mg, about 10 mg to 40 mg, or more than about 40 mg.

In some embodiments, the compound of Formula (II), or a pharmaceutically acceptable salt thereof, is in an amount of about 10 mg to about 40 mg.

In some embodiments, the compound of Formula (II), or a pharmaceutically acceptable salt thereof, is in an amount of about 5 mg.

In some embodiments, the compound of Formula (II), or a pharmaceutically acceptable salt thereof, is in an amount of about 10 mg.

In some embodiments, the compound of Formula (II), or a pharmaceutically acceptable salt thereof, is in an amount of about 20 mg.

In some embodiments, the compound of Formula (II), or a pharmaceutically acceptable salt thereof, is in an amount of about 40 mg.

In some embodiments, the composition further comprises at least one further active ingredient, wherein said at least one further active ingredient is a compound which normalizes lipid metabolism.

In some embodiments, the composition comprises at least one further active ingredient, wherein said at least one further active ingredient is selected from the group comprising one or more antidiabetics, hypoglycemic active ingredients, HMGCoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbents, LDL receptor inducers, ACAT inhibitors, antioxidants, lipoprotein lipase inhibitors, ATP-citrate lyase inhibitors, squalene synthetase inhibitors, lipoprotein (a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose-1,6-bisphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY agonists, MC4 agonists, orexin agonists, H3 agonists, TNF agonists, CRF agonists, CRF BP antagonists, urocortin agonists, β3 agonists, CB 1 receptor antagonists, MSH (melanocyte-stimulating hormone) agonists, CCK agonists, serotonin reuptake inhibitors, mixed serotoninergic and noradrenergic compounds, 5HT agonists, bombesin agonists, galanin antagonists, growth hormones, growth hormone-releasing compounds, TRH agonists, uncoupling protein 2 or 3 modulators, diphenylazetidinone derivatives, leptin agonists, DA agonists (bromocriptine, Doprexin), lipase/amylase inhibitors, PPAR modulators, RXR modulators or TR-β agonists or amphetamines.

In some embodiments, a composition or unit dosage form described herein is administered as an emulsion, a solution, a suspension, a syrup, a slurry, a dispersion, a colloid, a dissolving tablet, a dissolving wafer, a capsule, a gel capsule, a solid, a semi-solid, a solid forma gel, a gel matrix, a cream, a paste, a tablet, a granule, a sachet, a powder, or the like.

In some embodiments, the composition is in solid dosage form.

In some embodiments, the solid dosage form is a capsule, a pill, a cachet, a tablet, a granule, or powder.

In some embodiments, the composition is stable at room temperature and 60% relative humidity (RH) for at least 9 weeks.

In some embodiments, the composition is stable at 40° C. and 75% RH for at least 8 weeks.

In some embodiments, the composition is stable for at least about 3, 4, 6, 8, 10, 12, 14, 16, 18, 20, 24, 28, 32, 36, 40, 44, 48, 52, 58, or 64 months. In some embodiments, the composition is stable for about 3, 4, 6, 8, 10, 12, 14, 16, 18, 20, 24, 28, 32, 36, 40, 44, 48, 52, 58, or 64 months

In some embodiments, the composition is stable for at least 3 months.

In some embodiments, the composition is stable for at least 4 months.

In some embodiments, the composition is stable for at least 36 months.

### Methods of Treatment

Provided herein are embodiments directed to a method of treating a cholestatic liver disease in a subject in need thereof, wherein the method comprises administering to the subject a therapeutically effective amount of the pharmaceutical composition described above.

In the embodiments, the cholestatic liver disease is a pediatric cholestatic liver disease.

In the embodiments, the cholestatic liver disease is an adult cholestatic liver disease.

In the embodiments, the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

In the embodiments, the cholestatic liver disease is selected from the group consisting of ALGS, PFIC, BA, ICP, BRIC, PSC, and PBC.

In the embodiments, the cholestatic liver disease is PSC.

In the embodiments, the cholestatic liver disease is PBC.

In the embodiments, the cholestatic liver disease is ICP.

In the embodiments, the cholestatic liver disease is characterized by pruritus.

In the embodiments, the subject has elevated total serum bile acids (sBA) prior to the administration of the pharmaceutical composition.

In the embodiments, the compound of Formula (I) is an ileal bile acid transporter (IBAT) inhibitor.

In the embodiments, the compound of Formula (I) has the structure of the compound of Formula (II) that is an ileal bile acid transporter (IBAT) inhibitor.

Also provided herein are embodiments directed to a method of treating hyperlipidemia in a subject in need thereof, wherein the method comprises administering to the subject a therapeutically effective amount of the pharmaceutical composition described above.

Further provided herein are embodiments directed to a method of lowering the serum cholesterol level in a subject in need thereof, wherein the method comprises administering to the subject a therapeutically effective amount of the pharmaceutical composition described above.

Additionally provided herein are embodiments directed to a method of treating arteriosclerosis in a subject in need thereof, wherein the method comprises administering to the subject a therapeutically effective amount of the pharmaceutical composition described above.

Additionally provided herein are embodiments directed to a method of treating Syndrome X in a subject in need thereof, wherein the method comprises administering to the subject a therapeutically effective amount of the pharmaceutical composition described above.

The pharmaceutical compositions of this invention can be administered by a variety of routes including oral, rectal, intraocular, transdermal, subcutaneous, intravenous, intramuscular, intraperitoneal, intradermal, directly into cerebrospinal fluid, intratracheal, and intranasal. In some embodiments, the composition is administered to the subject orally.

### Methods of Producing the Pharmaceutical Composition and Kits

Provided herein are embodiments directed to a method of producing the pharmaceutical composition described above comprising the steps of: combining a therapeutically effective amount of the compound of Formula (I)), or a pharmaceutically acceptable salt thereof, and one or more excipients selected from the group consisting of:
(i) a diluent or a filler,
(ii) a disintegrant,
(iii) a channeling agent,
(iv) a glidant, and
(v) a lubricant
to form an admixture; mixing the admixture; and filling in a capsule shell or compacting the admixture to form the pharmaceutical composition.

Further provided herein are embodiments directed to a kit for treating cholestatic liver disease in a subject in need thereof, wherein the kit comprises at least one unit dosage of a therapeutically effective amount of the composition described above and instructions for the administration thereof.

In some embodiments, the kit is for treating the cholestatic liver disease that is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

In some embodiments, the cholestatic liver disease is ALGS, PFIC, BA, ICP, BRIC, PSC, or PBC.

In some embodiments, the kit is for treating the cholestatic liver disease that is PSC.

In some embodiments, the kit is for treating the cholestatic liver disease that is PBC.

In some embodiments, the kit is for treating the cholestatic liver disease that is ICP.

Further provided herein are embodiments directed to a kit for treating hyperlipidemia in a subject in need thereof, wherein the kit comprises a therapeutically effective amount of the composition described above.

Further provided herein are embodiments directed to a kit for lowering the serum cholesterol level in a subject in need thereof, wherein the kit comprises a therapeutically effective amount of the composition described above.

Further provided herein are embodiments directed to a kit for treating arteriosclerosis in a subject in need thereof, wherein the kit comprises a therapeutically effective amount of the composition described above.

Further provided herein are embodiments directed to a kit for treating Syndrome X in a subject in need thereof, wherein the kit comprises a therapeutically effective amount of the composition described above.

### EXAMPLES

The following examples are provided to further describe some of the embodiments disclosed herein. The examples are intended to illustrate, not to limit, the disclosed embodiments.

### Example 1. Excipient Compatibility

Binary formulations of volixibat potassium were prepared at either 1:1 or 10:1 ratio with the excipients outlined in Table 1.1. Powder grade excipients were utilized to maximize surface area interactions between excipient and drug substance.

Each formulation was prepared at 300 mg total sample size. Volixibat and each excipient was weighed into glass vials and mixed via vortex. A single vial of each formulation was stored loosely capped at two storage conditions: ambient/60% RH and 40° C./75% RH. Humidity control was obtained by using a saturated salt solution in a sealed container. The single vial of each formulation was sampled for all three scheduled time points (T=2, 4, and 8 weeks). A standard solution of volixibat DS prepared at nominal concentration in the analytical diluent served as the T=0 data point. The neat, loose drug substance powder was stored in the same vial configuration to serve as a control at each storage condition.

At the specified time points, approximately 50 mg of each formulation was sampled and diluted with analytical diluent in either 50 mL (for 1:1 formulations) or 100 mL (for 1:10 formulations and neat DS) to target nominal analytical concentration of 0.5 mg/mL volixibat. HPLC analysis was used based on the parameters described above under HPLC Parameters (Gradient Elution).

TABLE 1.1
Selected Excipients and Target Ratios

| | | | | Target | Volixibat | Excipient |
|---|---|---|---|---|---|---|
| Sample | Proposed | | | Volixibat:Excipient | weight | weight |
| # | Excipient | Vendor/Grade | Function | Ratio | (mg) | (mg) |
| 1 | Lactose anhydrous | Kerry/Sheffield | Diluent/ | 1:1 | 150 | 150 |
| | | Lactose | compression | | | |
| | | anhydrous 60M | aid | | | |
| 2 | Microcrys talline | DuPont/FMC | Diluent/ | | | |
| | cellulose | Avicel PH101 | compression | | | |
| | | | aid | | | |
| 3 | Mannitol | Roquette Pearlitol | Diluent/ | | | |
| | | 50C (RAI) sieved | compression | | | |
| | | 40 mesh | aid | | | |
| 4 | Starch 1500 | Dow/Colorcon | Diluent/ | | | |
| | | Starch 1500 | compression | | | |
| | | | aid | | | |
| 5 | Croscarmellose | DuPont/FMC | Disintegrant | 10:1 | 270 | 30 |
| | sodium | AcDiSol | | | | |
| 6 | Sodium starch | JRS Pharma | Disintegrant | | | |
| | glycolate | | | | | |
| 7 | Silicon dioxide | Cabot | Glidant | | | |
| | | Cab-O-Sil-M-5P, | | | | |
| | | sieved 40 mesh | | | | |
| 8 | Magnesium | Spectrum NF, | Lubricant | | | |
| | stearate | sieved 40 mesh | | | | |
| 9 | Sodium stearyl | Spectrum | Lubricant | | | |
| | fumarate | | | | | |
| 10 | Sodium chloride | Spectrum | Channeling | | | |
| | | | agent | | | |
| 11 | PEG 1500 | Fluka or Sigma- | Channeling | | | |
| | | Aldrich | agent | | | |
| 12 | Neat volixibat DS | Lot 7U001 | Control Sample | NA | 300 | NA |

Potency of samples fluctuated significantly at each time point. This variability is expected given variability in sampling, possible water uptake due to open-dish conditions, and impact to dilutions from insoluble excipients. Potency data was not used to trend stability but was reported as supporting data for evaluation alongside the impurity profiles.

Purity remained stable for all samples at both storage conditions; samples stored at 40° C./75% RH had slightly more growth in related substances. The overall peak purity for volixibat remained stable for all samples at both storage conditions, indicating acceptable compatibility with all tested excipients.

TABLE 1.2
Potency Results for Samples Stored at RT/60% RH

| | % LC Volixibat | | |
|---|---|---|---|
| Sample Name | T = 2-weeks | T = 4 weeks | T = 9 weeks |
| Lactose Anhydrous | 91.9 | 107.1 | 98.7 |
| MCC | 97.4 | 101.9 | 98.6 |
| Mannitol | 106.2 | 118.5 | 108.8 |
| Starch 1500 | 101.9 | 106.0 | 103.6 |
| CCS | 93.3 | 104.1 | 98.2 |
| SSG | 89.1 | 105.4 | 101.0 |
| SiO2 | 102.0 | 106.4 | 100.1 |
| MgSt | 100.2 | 106.7 | 100.6 |
| SSF | 100.7 | 105.3 | 100.3 |
| NaCl | 101.5 | 99.3 | 91.0 |
| PEG 1500 | 93.5 | 101.4 | 94.4 |
| Volixibat DS | 100.9 | 107.7 | 97.1 |

TABLE 1.3
Potency Results for Samples Stored at 40° C./75% RH

| | % LC Volixibat | | |
|---|---|---|---|
| Sample Name | T = 2-weeks | T = 4 weeks | T = 8 weeks |
| Lactose Anhydrous | 90.2 | 96.2 | 91.9 |
| MCC | 98.4 | 102.9 | 96.2 |
| Mannitol | 104.8 | 99.1 | 94.3 |
| Starch 1500 | 95.7 | 103.9 | 100.6 |
| CCS | 95.3 | 100.8 | 100.0 |
| SSG | 94.5 | 100.8 | 94.3 |
| SiO2 | 100.1 | 105.3 | 100.5 |
| MgSt | 99.7 | 103.2 | 98.6 |
| SSF | 97.9 | 105.0 | 99.1 |
| NaCl | 78.5 | 80.8 | 91.0 |
| PEG 1500 | 93.1 | 99.0 | 97.7 |
| Volixibat DS | 93.2 | 105.1 | 98.3 |

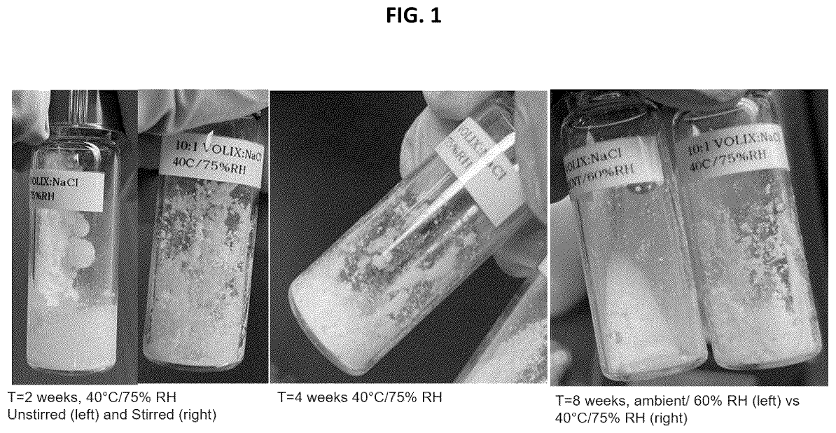

The mixture of NaCl:volixibat had significant water uptake at the 40° C./75% RH storage condition resulting in agglomeration and stickiness for that sample (FIG. 1). The RT/60% RH sample of NaCl:volixibat did not exhibit the physical agglomeration or significant growth of RRT 0.86.

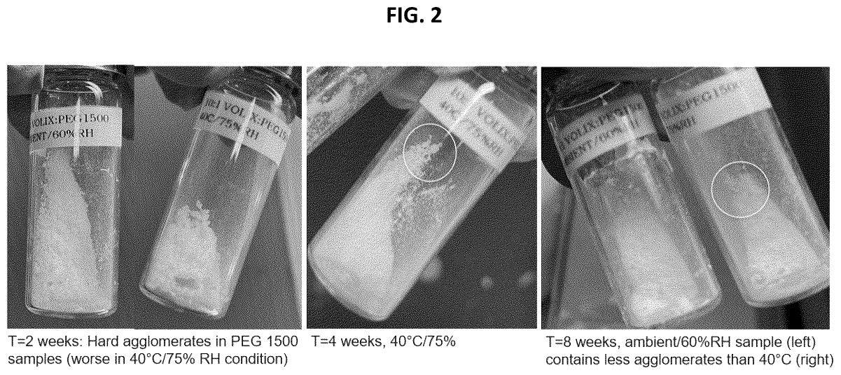

The PEG 1500:volixibat formulations stored at 40° C./75% RH exhibited some formation of hard agglomerates by 2 weeks (see FIG. 2) but remained consistent throughout the remainder of the study. This agglomeration was not as apparent in the RT/60% RH sample. There was no change in the related substance profile for PEG1500:volixibat over 8 weeks for either storage condition.

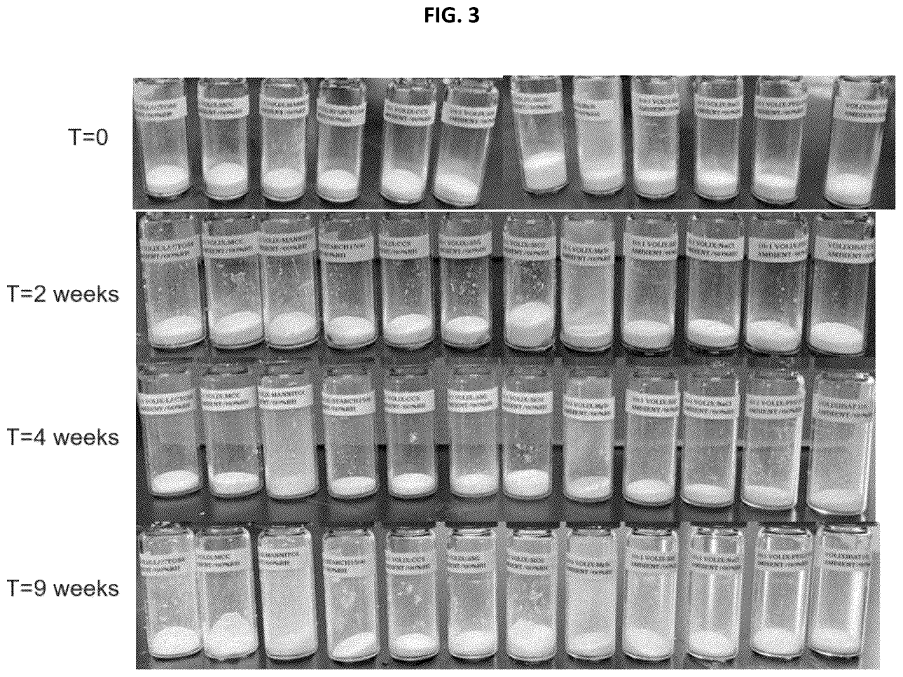

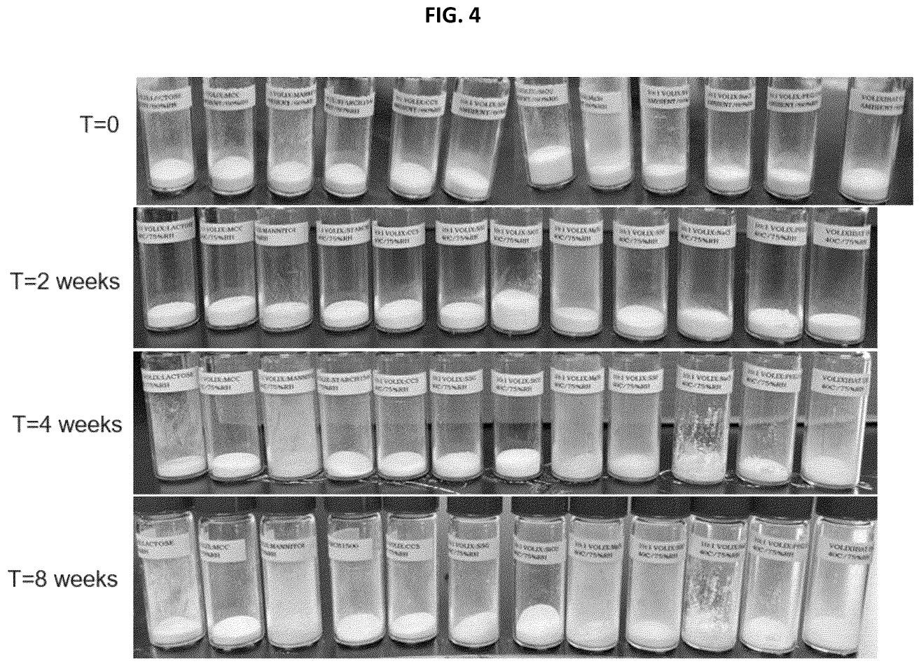

All other samples remained white, loose powders over the course of the study. No color change or significant agglomeration was observed for either storage condition (FIGS. 3-4).

### Example 2. Formulation Development

### Overview of Formulations

TABLE 2.1
Composition of Formulations

| | | Formulation | Formulation | Formulation | Formulation | Formulation | Formulation |
|---|---|---|---|---|---|---|---|
| | | 1 | 2 | 3 | 4 | 5 | 6 |
| Component | Function | (%) | (%) | (%) | (%) | (%) | (%) |
| Volixibat (VXT) | DS | 15.00 | 15.00 | 14.96 | 14.96 | 7.50 | 7.50 |
| potassium | | | | | | | |
| MCC Avicel | Diluent/ | 44.43 | 44.43 | 48.04 | 58.04 | 56.00 | 46.00 |
| PH102 | Compression | | | | | | |
| | Aid | | | | | | |
| Lactose | Diluent/ | 14.29 | — | 25.00 | 15.00 | 15.00 | 40.00 |
| Monohydrate | Compression | | | | | | |
| SD 316 | Aid | | | | | | |
| Mannitol 100 | Diluent/ | — | 14.29 | — | — | — | — |
| SD | Compression | | | | | | |
| | Aid | | | | | | |
| Sodium chloride | Channeling | 14.29 | 14.29 | 5.00 | 5.00 | 15.00 | — |
| | Agent | | | | | | |
| Sodium starch | Disintegrant | 10.00 | 10.00 | 5.00 | 5.00 | 5.00 | 5.00 |
| glycolate | | | | | | | |
| Silicon dioxide | Glidant | 1.00 | 1.00 | 1.00 | 1.00 | 1.00 | 1.00 |
| Magnesium | Lubricant | 1.00 | 1.00 | 1.00 | 1.00 | 0.50 | 0.50 |
| stearate | | | | | | | |

| | | | | | | |
|---|---|---|---|---|---|---|
| Total % | 100 | 100 | 100 | 100 | 100 | 100 |

TABLE 2.2
Formulation Flow Properties

| | | Existing | | | | | | |
|---|---|---|---|---|---|---|---|---|
| | Volixibat | Formulation | Formulation | Formulation | Formulation | Formulation | Formulation | Formulation |
| Parameter | DS | (20 mg) | 1 | 2 | 3 | 4 | 5 | 6 |
| Bulk | 0.54 | 0.51 | 0.46 | 0.43 | 0.42 | 0.42 | 0.43 | 0.45 |
| Density | | | | | | | | |
| (g/mL) | | | | | | | | |
| Tapped | 0.69 | 0.69 | 0.63 | 0.61 | 0.59 | 0.57 | 0.59 | 0.60 |
| Density | | | | | | | | |
| (g/mL) | | | | | | | | |
| Hausner | 1.27 | NA | 1.37 | 1.41 | 1.40 | 1.36 | 1.38 | 1.31 |
| Ratio | | | | | | | | |
| Angle of | NT | NT | 30.6 | 31.3 | 27.4 | 23.2 | 32.5 | 31.3 |
| Repose | | | | | | | | |

NT = Not tested

Formulations 1 and 2 were created 15% w/w volixibat potassium (no correction factor applied). These two formulations were first tested as 20 mg capsules. Flow properties and dissolution results were similar for both formulations, so the Formulation 1 diluent system was selected. Formulations 3-4 lowered the level of channeling agent and disintegrant and accommodated the decrease with an increase in either lactose (Formulation 3) or Avicel (Formulation 4). Again, there was not a significant impact to the flow or dissolution properties of 20 mg capsules. Formulation 3 and 4 dissolution profiles consistent with one another and with Formulation 1. This confirmed the level of sodium chloride and sodium starch glycolate could be lowered in the final formulation if needed. Formulation 3 (increased lactose) had lower release than Formulation 4 (increased Avicel) in phosphate buffer so for Formulation 5 an increase in Avicel was favored when accommodating for formulation modifications.

Formulation 5 was manufactured with a lower concentration of Volixibat Potassium in the blend. The magnesium stearate was also lowered in Formulation 5. Formulation 5 exhibited acceptable dissolution and flow properties and was also evaluated for segregation and changes to dissolution from over-lubrication. Content uniformity maintained low % RSD when tested at 0 and 7 days after formulation creation (no segregation observed) and over-mixing of the formulation did not impact the dissolution profile.

Formulation 6 was formulated without NaCl and 20 mg capsules were tested in all the same set of media. While increased Avicel (Formulation 4) resulted in higher release in phosphate pH 6.8, the dissolution results in acidic media (acetate pH 4.5) were improved with higher lactose (Formulation 3). Formulation 6 did not reach full release in 0.1N HCl despite solubility data indicating sink conditions were met. Further studies concluded that volixibat extraction is limited in 0.1N HCl even without excipients present (Example 2). The acidic media was changed to 0.01N HCl. Formulation 6 was still evaluated in each of the media (0.01N HCl, phosphate buffer, and acetate buffer) to inform the impact of NaCl in the formulation.

### Formulations 1-2: 40 mg Target Dose, Evaluation of Diluent System

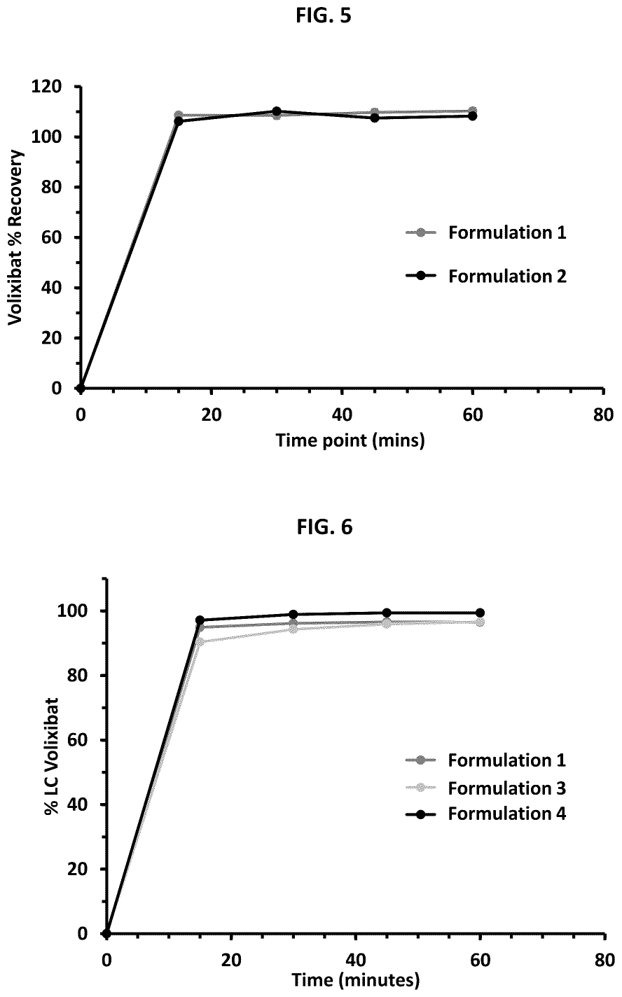

Formulations 1 and 2 were created with 15% w/w volixibat potassium. The diluent system in Formulation 1 was representative of the existing volixibat capsule formulation, while Formulation 2 had mannitol instead of lactose. Historical dissolution data for volixibat capsules indicated the higher dosage strength (20 mg) exhibited gelling and slowed release. For each formulation, capsules were filled using tamping or loose-fill to determine impact of tamping on capsule release. Dissolution testing was performed in 900 mL phosphate buffer, pH 6.8. Both formulations exhibited immediate dissolution, with capsule contents dispersing within 7-10 minutes. No gelling was observed, and there was not a significant difference in release between formulations or between tamped and loose-filled capsules. An overlay of the dissolution profile for Formulation 1 vs Formulation 2 (n=6, both tamped and bulk capsules included) can be found in FIG. 5. Since Formulations 1 and 2 were comparable, the diluent system was maintained as Avicel:lactose for consistency with the existing capsule formulation. Subsequent formulations 3-4 evaluated decreased channeling agent (NaCl) and disintegrant (sodium starch glycolate).

### Formulations 3-4: Optimizing Channeling Agent and Disintegrant

Formulations 3-4 were prepared with decreased levels of NaCl and sodium starch glycolate. To compensate for the decrease in these components, Formulation 3 had increased ratio of Avicel while Formulation 4 had increased lactose. Additionally, to reach the % w/w volixibat Potassium, the potency (0.9165) and salt correction (0.9549) were included in calculations to determine target weight of volixibat potassium during formulation preparation. Bulk/tapped density and angle of repose showed each of these formulations had similar flow properties as Formulations 1-2 (Table 2.1).

TABLE 2.3
Formulation Composition

| | Formulation 3 (40 mg dose) | | | Formulation 4 (40 mg dose) | | |
|---|---|---|---|---|---|---|
| | | mg/ | g/ | | mg/ | g/ |
| Component | (%) | capsule | batch | (%) | capsule | batch |
| Volixibat Potassium | 14.96 | 41.89 | 14.96 | 14.96 | 41.89 | 14.96 |
| Microcrystalline | 48.04 | 134.51 | 48.04 | 58.04 | 162.51 | 58.04 |
| cellulose | | | | | | |
| Lactose anhydrous | 25.00 | 70.00 | 25.00 | 15.00 | 42.00 | 15.00 |
| Mannitol | NA | NA | NA | NA | NA | NA |
| Sodium chloride | 5.00 | 14.00 | 5.00 | 5.00 | 5.00 | 5.00 |
| Sodium starch | 5.00 | 14.00 | 5.00 | 5.00 | 5.00 | 5.00 |
| glycolate | | | | | | |
| Silicon dioxide | 1.00 | 2.80 | 1.00 | 1.00 | 1.00 | 1.00 |
| Magnesium stearate | 1.00 | 2.80 | 1.00 | 1.00 | 1.00 | 1.00 |
| Total | 100.00% | 280 mg | 100.0 g | 100.00% | 280 mg | 100.0 g |

Dissolution of the 40 mg dose was tested in size 0 gelatin capsules in phosphate buffer (pH 6.8) to evaluate impact of decreased channeling agent and disintegrant. The fill weight for these capsules was 280 mg. At this time, the dissolution volume was changed from 900 mL to 500 mL to match the current analytical method. Given the high aqueous solubility of volixibat (>200 mg/mL at 25° C.) sink conditions were still met for 40 mg capsules in 500 mL (0.08 mg/mL). Volixibat Formulation 1 was also re-tested in 500 mL dissolution media as a control, but the fill weight for 40 mg capsules was adjusted to 304.7 mg to include the potency correction.

Formulations 1, 3, and 4 exhibited similar dissolution in 500 mL phosphate pH 6.8 media (FIG. 6). Formulation 3 (with increased lactose) had the lowest overall recovery and the highest variability. For Formulation 5, lactose levels were maintained at 15% and Avicel was increased to accommodate formulation changes. There was no impact to the visual capsule dissolution or the overall dissolution rate for Formulations 3 and 4, confirming that the channeling agent and disintegrant in the formulation could be lowered if necessary without impacting the 40 mg capsule release profile.

### Formulation 5: 20 mg Target Dose

Formulation 5 was designed with similar composition to Formulation 1 but with lower drug loading as well as a decrease in sodium starch glycolate (disintegrant) from 10% to 5%. NaCl (channeling agent) was maintained at the original level of 15%. Magnesium stearate was decreased to 0.5% based on over-lubrication issues that had been observed historically for other formulations of volixibat. The % w/w for Avicel was increased to accommodate weight changes for the other components based on the results observed for Formulations 3-4 (slightly improved dissolution with increased Avicel vs lactose). As with Formulations 3-4, the amount of volixibat potassium added was corrected to accommodate salt and purity factors. See Table 2.4.

TABLE 2.4
Formulation 5 Composition

| | | | Formulation 5 (5 mg and 20 dose) | | | |
|---|---|---|---|---|---|---|
| | | | | 5 mg/ | 20 mg/ | g/ |
| Component | Vendor/Grade | Function | (%) | capsule | capsule | batch |
| Volixibat Potassium | Lot 7U001 | DS | 7.5 | 5.236 | 20.944 | 7.5 |
| Microcrystalline | DuPont/FMC | Diluent/ | 56 | 39.214 | 156.856 | 56 |
| cellulose | Avicel PH102 | Compression | | | | |
| | | Aid | | | | |
| Lactose anhydrous | Kerry/Sheffield | Diluent/ | 15 | 10.5 | 42.0 | 15 |
| | Lactose | Compression | | | | |
| | Monohydrate | Aid | | | | |
| | SD 316 | | | | | |
| Sodium chloride | Oakwood | Channeling | 15 | 10.5 | 42.0 | 15 |
| | Chemicals, ACS | Agent | | | | |
| Sodium starch | JRS Pharma | Disintegrant | 5 | 3.5 | 14.0 | 5 |
| glycolate | (Explotab) | | | | | |
| Silicon dioxide | Cabot | Glidant | 1 | 0.7 | 2.8 | 1 |
| | Cab-O-Sil-M-5P | | | | | |
| Magnesium | Spectrum | Lubricant | 0.5 | 0.35 | 1.4 | 0.5 |
| stearate | | | | | | |

| | | | | |
|---|---|---|---|---|
| Total | 100.00% | 70.00 mg | 280.00 mg | 100.0 g |

### Formulation 5 Evaluated for Segregation and Over-Lubrication

Formulation 5 was used to evaluate potential segregation as well as over-lubrication studies. The formulation was tested for assay, content unity (“CU”), and dissolution at T=0, then held for 7 days undisturbed (stored in a glass jar sealed within an aluminum bag). After 7 days, CU was re-tested to evaluate for segregation. CU was tested by sampling 280 mg each from 10 different locations in the jar (total batch size in jar was 50 g). The formulation was then blended in the turbula for an additional 5 minutes to create over-lubrication. The over-mixed formulation was filled into size 0 capsules and tested for dissolution in 500 mL phosphate buffer (pH 6.8). No filtration step was used for the T=0 or T=7-day dissolution testing.

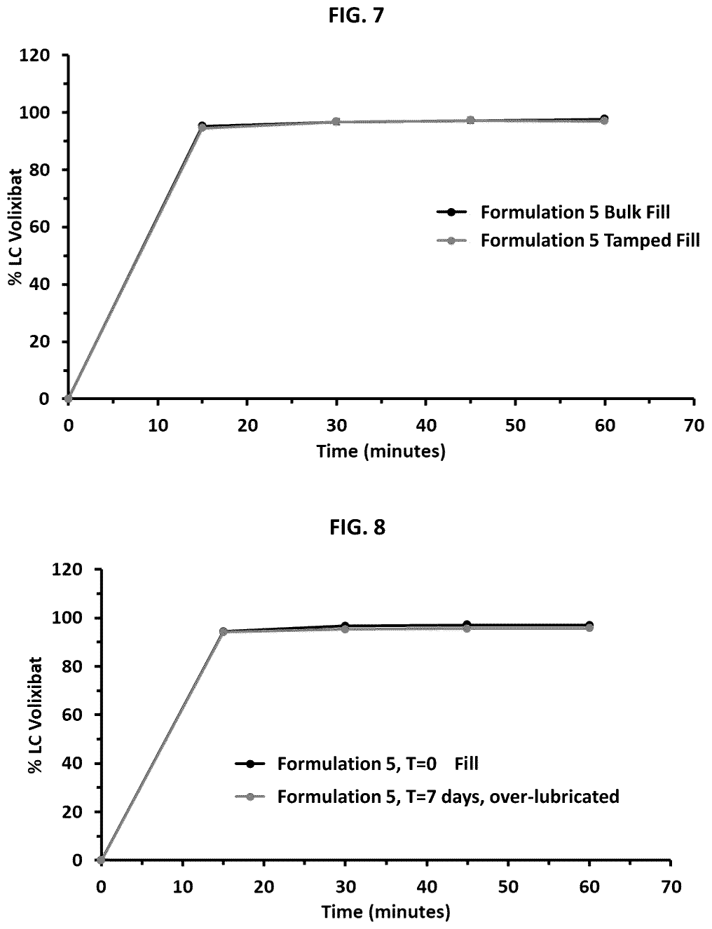

The T=0 assay for Formulation 5 resulted 97.30% LC and 99.85% purity. T=0 dissolution was tested on a total of 6 capsules, n=3 bulk-fill and n=3 tamped-fill. Consistent with the previous formulations, the level of tamping did not impact the capsule dissolution profile: capsules dissolved and contents dispersed within 10 minutes, and most of the release was obtained by the 15-minute time point (FIG. 7).

Table 2.5 includes CU results at T=0 and after 7-days sitting undisturbed on the benchtop. CU samples at T=0 and T=7 days had a low RSD of ˜3%. Acceptance Value (AV) as calculated per instructions in USP<905> was 4.78 at T=0 and 7.50 at T=7 days. Despite the slight increase in AV, both values were below the recommended limit of 15 outlined in USP<905>, and the similar RSD at both time points indicate the batch uniformity was not significantly changed after 7 days storage.

After CU was tested on the formulation at T=7 days, the formulation was mixed in the turbula for an additional 5 minutes to over-lubricate the blend. Capsules were filled to 20 mg dose (tamped fill) and evaluated for dissolution in phosphate pH 6.8. The dissolution was consistent with T=0 results, indicating the additional mixing did not significantly over-lubricate the blend. See FIG. 8. It is surprising and unexpected that the lower magnesium stearate level may be beneficial in the finalized formulation.

TABLE 2.5
Formulation 5, Content Uniformity T = 0 vs T = 7 Days

| Sample | T = 0% LC | T = 7 days % LC |
|---|---|---|
| CU 1 | 92.9 | 101.4 |
| CU 2 | 93.2 | 97.7 |
| CU 3 | 94.2 | 104.9 |
| CU 4 | 99.9 | 97.4 |
| CU 5 | 91.6 | 103.0 |
| CU 6 | 93.4 | 101.6 |
| CU 7 | 95.9 | 104.9 |
| CU 8 | 100.6 | 104.4 |
| CU 9 | 95.0 | 98.4 |
| CU 10 | 99.0 | 104.3 |
| Average | 95.6 | 101.8 |
| % RSD | 3.4 | 3.0 |
| AV (per USP <905>) | 4.78 | 7.50 |

### Example 3. Dissolution in Various Media

Formulation 1, 3, 4, and 5 were tested for dissolution in three different media: phosphate pH 6.8, acetate pH 4.5, and 0.1N HCl.

### Dissolution in Acetate Buffer pH 4.5

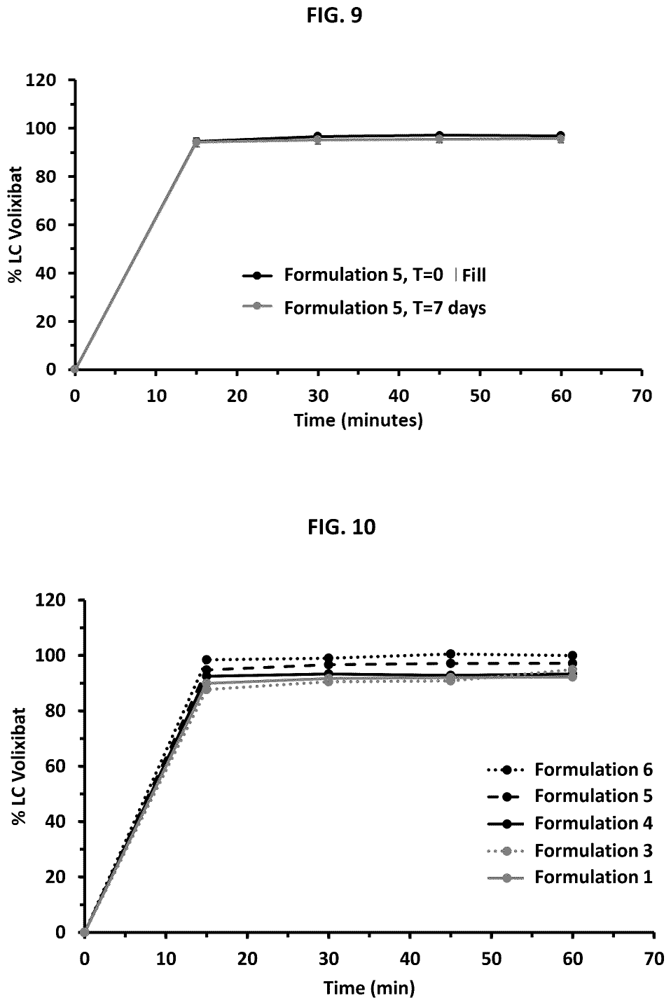

Overlay of dissolution results in acetate buffer pH 4.5 are included in FIG. 9. This release was consistent with visual observations during testing: capsules fully dissolved, and contents readily dispersed in 10-15 minutes. No agglomeration or gelling was observed for any of the formulations in acetate buffer pH 4.5.

### Dissolution in Phosphate Buffer pH 6.8

Formulations 1, 3, 4, 5, and 6 were filled to 20 mg target dose in size 1 capsules and tested for dissolution in 500 mL of phosphate buffer pH 6.8.

As with acetate buffer, the dissolution profile was similar for all formulations, but Formulations 5 and 6 reached the highest overall release (FIG. 10).

TABLE 3.1
Assay of Each Formulation

| | Approx. age of | % LC | % Area |
|---|---|---|---|
| | formulation | (Assay) | (Purity) |
| Formulation 1 | 6 months | 102 | 99.89 |
| Formulation 3 | 4 months | 104 | 99.85 |
| Formulation 4 | 4 months | 102 | 99.87 |
| Formulation 5 | 3 months | 99 | 99.86 |

### Dissolution in Acidic Media

Initially an evaluation of all formulations in 0.1N HCl was investigated.

TABLE 3.2
Formulation 5 in 0.1N HCl
% LC Volixibat

| Sample | 15 min | 30 min | 45 min | 60 min |
|---|---|---|---|---|
| V1 | 33.5 | 32.2 | 35.8 | 36.4 |
| V2 | 28.4 | 34.0 | 36.7 | 37.6 |
| V3 | 30.4 | 36.0 | 36.8 | 38.6 |
| Avg. | 30.8 | 34.1 | 36.4 | 37.5 |
| Std Dev | 2.6 | 1.9 | 0.6 | 1.1 |

TABLE 3.3
Formulation 6 in 0.1N HCl
% LC Volixibat

| Sample | 15 min | 30 min | 45 min | 60 min |
|---|---|---|---|---|
| V1 | 29.4 | 35.5 | 38.7 | 39.8 |
| V2 | 27.8 | 34.2 | 36.4 | 37.8 |
| V3 | 27.7 | 35.5 | 38.0 | 39.0 |
| Avg. | 28.3 | 35.0 | 37.7 | 38.9 |
| Std Dev | 0.9 | 0.7 | 1.2 | 1.0 |

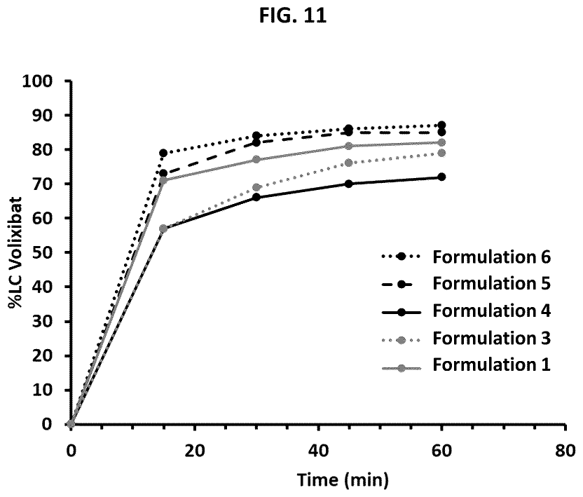

Consistent with the dissolution testing in phosphate and acetate buffers, dissolution in 0.01N HCl was carried out using each formulation filled to 20 mg target dose in size 1 gelatin capsules. The media fill was 500 mL, clear vessels were used, and neutralization was performed immediately after each pull (0.5 mL of 0.01N NaOH added to 0.5 mL of dissolution sample) to limit degradation between sampling and analysis. Results are outlined in FIG. 11 and Table 3.4.

### Dissolution Results Summary

Results for 60-min dissolution (average of 6 vessels) for each formulation in the three media is included in Table 3.4. In phosphate buffer pH 6.8, all formulations resulted in similar capsule release profile and final % recovery for volixibat. Differences between formulations became more apparent in lower pH media. All formulations had an overall slower release profile and lower final recovery in 0.01N HCl but Formulation 4 had significantly lower overall recovery. Formulations 1, 3, and 4 have higher drug loading (15% volixibat as opposed to 7.5% in Formulations 5 and 6). Formulations 3 and 4 also had lower levels of sodium chloride (5% as opposed to 15% in other formulations), with Formulation 3 having higher proportion of lactose and Formulation 4 having a higher level of Avicel to accommodate the lower NaCl levels. Formulation 4 had higher recovery than Formulation 3 in the nominal media (phosphate pH 6.8) when tested at both 20 mg and 40 mg doses, so Formulation 5 implemented higher Avicel loading to accommodate formulation changes. While Formulation 4 performed better than Formulation 3 in phosphate pH 6.8, it had lower recovery in both acetate and 0.01N HCl, suggesting increase in lactose may be beneficial for volixibat dissolution at lower pH values. These results informed the creation of Formulation 6, which removed NaCl and increased lactose to accommodate the change with the goal of improving dissolution in the desired 0.1N HCl media. Formulation 6 did not show improvement in dissolution in 0.1N HCl, and the acidic media used for the dissolution screening was changed to 0.01N HCl. Formulation 5 maintained the higher Avicel:lactose ratio based on improved dissolution in the nominal phosphate pH 6.8 buffer. As indicated in Table 3.4, Formulations 5 and 6 performed similarly across the three media tested.

TABLE 3.4
Overall Dissolution at 60 min for Each Formulation/Media
20 mg Capsules 60 min Avg Results (% LC)

| Formulation | 1 | 3 | 4 | 5 | 6 |
|---|---|---|---|---|---|
| Acetate Buffer pH 4.5 | 92 | 94 | 89 | 95 | 98 |
| Phosphate Buffer pH 6.8 | 97 | 97 | 99 | 97 | 100 |
| 0.01N HCl | 82 | 79 | 72 | 85 | 87 |

### Example 4. Studies of Formulation 7

Formulation 7 was developed with 6.55% VLX and was filled into size 4 capsules and size 0 capsules (see Table 4.1). This resulted in 80 mg fill for the 5 mg dose in size 4 capsules and 320 mg fill for the 20 mg dose in size 0 capsules. Pro-filling Formulation 7 was successful at these fill targets and capsule sizes. Weight sorting was performed on all capsules with a tolerance of +/−5% of the target fill weight.

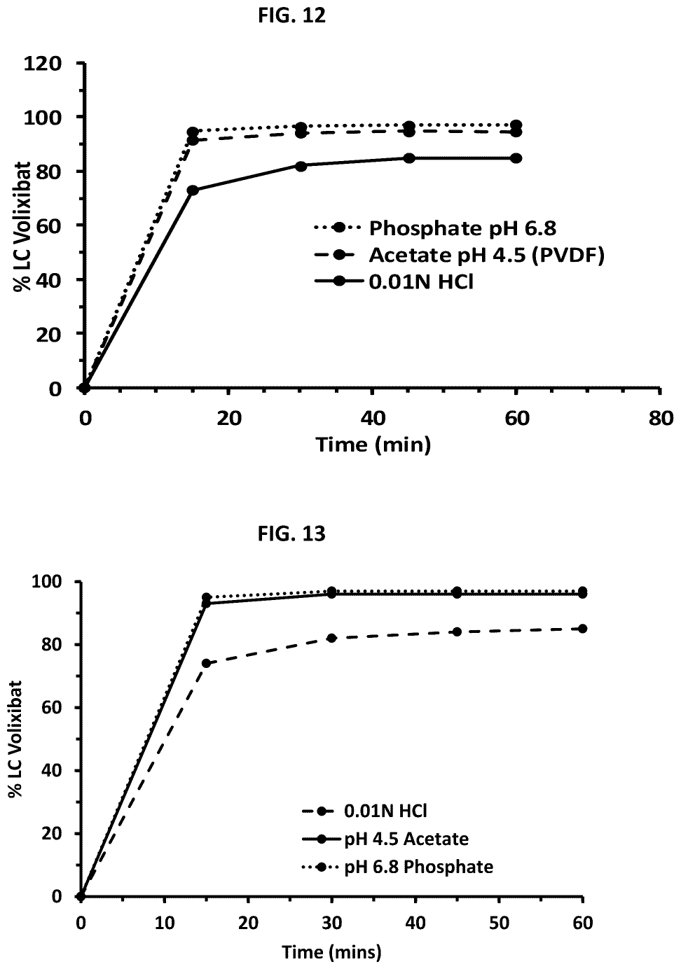

The flow properties and dissolution results for Formulation 7 are included in Table 4.2 and FIG. 12. Dissolution in each media was also very consistent for Formulation 5 and Formulation 7, with ˜95% release in phosphate pH 6.8 and acetate pH 4.5, and only 85% release in 0.01N HCl.

TABLE 4.1
Formulation 7 for Higher Capsule Fill

| | | Formulation 7 | | |
|---|---|---|---|---|
| | | | 5 mg/ | 20 mg/ |
| Component | Function | % | capsule | capsule |
| Volixibat (VXT) potassium | DS | 6.55% | 5.24 | 20.96 |
| MCC Avicel PH102 | Diluent/ | 56.95% | 45.56 | 182.24 |
| | Compression | | | |
| | Aid | | | |
| Lactose Monohydrate SD | Diluent/ | 15.0% | 12.00 | 48.00 |
| 316 | Compression | | | |
| | Aid | | | |
| Sodium chloride | Channeling | 15.0% | 12.00 | 48.00 |
| | Agent | | | |
| Sodium starch glycolate | Disintegrant | 5.0% | 4.00 | 16.00 |
| Silicon dioxide | Glidant | 1.0% | 0.80 | 3.20 |
| Magnesium stearate | Lubricant | 0.5% | 0.40 | 1.60 |

| | | | |
|---|---|---|---|
| Total | 100.00% | 80.00 | 320.00 |

TABLE 4.2
Flow Properties of Formulation 7

| Parameter | Formulation 5 | Formulation 7 |
|---|---|---|
| Bulk Density (g/mL) | 0.43 | 0.44 |
| Tapped Density (g/mL) | 0.59 | 0.60 |
| Hausner Ratio | 1.38 | 1.37 |
| Angle of Repose | 32.5 | 31.9 |

1-124. (canceled)

125. A pharmaceutical composition comprising a compound of Formula (I), 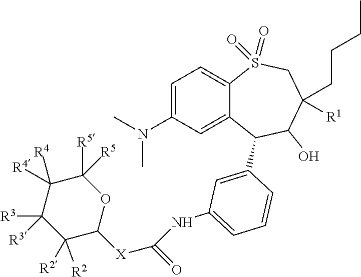
wherein:
X is NH;
R¹ is (C₁-C₆)-alkyl;
R² is OH;
R^{2′} is H;
R³, R^{3′}, R⁴, R^{4′}, R⁵, R^{5′}, are, independently H, Cl, Br, I, OH, (CH₂)—OH, CF₃, NO₂, N₃, CN, S(O)ₚ—R⁶, O—S(O)ₚ—R⁶, (C₁-C₆)-alkylene-S(O)ₚ—R⁶, (C₁-C₆)-alkylene-O—S(O)ₚ—R⁶, COOH, COO(C₁-C₆)alkyl, CONH₂, CONH(C₁-C₆)alkyl, CON[(C₁-C₆)alkyl]₂, (C₁-C₆)-alkyl, (C₂-C₆)alkenyl, (C₂-C₆)-alkynyl, O—(C₁-C₆)-alkyl, where one, more than one, or all hydrogen(s) in the alkyl radicals may be replaced by fluorine, phenyl, (CH₂)-phenyl, (CH₂)₂-phenyl, O-phenyl, O—(CH₂)ₘ-phenyl, (CH₂)—O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂;
wherein at least one of the radicals R³, R^{3′}, R⁴, R^{4′}, R⁵, and R^{5′} has the meaning of (C₁-C₆)-alkylene-O—S(O)ₚ—R⁶ and another has the meaning of —O—(CH₂)ₘ-phenyl, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂;
R⁶ is H, OH, (C₁-C₆)-alkyl, NH₂, NH(C₁-C₆)-alkyl or N((C₁-C₆)-alkyl)₂;
n is 2, 3, 4, 5 or 6;
m is 1, 2, 3, 4, 5 or 6;
p is 0, 1 or 2;
or a pharmaceutically acceptable salt thereof, and
(i) a diluent or a filler in an amount of about 55% to about 90% by weight of the total composition,
(ii) a disintegrant,
(iii) a channeling agent in an amount of about 5% to about 15% by weight of the total composition,
(iv) a glidant, and
(v) a lubricant.

126. The composition of claim 125, wherein the compound of Formula (I) has the structure of Formula (II), or a pharmaceutically acceptable salt thereof:

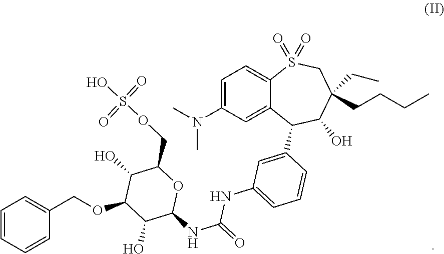 [SMILES: CCCC[C@]1(CC)CS(=O)(=O)c2ccc(N(C)C)cc2[C@@H](c2cccc(NC(=O)N[C@@H]3O[C@H](COS(=O)(=O)O)[C@@H](O)[C@H](OCc4ccccc4)[C@H]3O)c2)[C@H]1O]

127. The composition of claim 126, comprising the pharmaceutically acceptable salt of the compound of Formula (I), wherein the pharmaceutically acceptable salt is a potassium salt.

128. The composition of claim 125, wherein:
(i) the diluent or the filler is selected from the group consisting of dextrates, dextrin, dextrose, lactose, mannitol, sorbitol, cellulose, and modified celluloses, or a combination thereof;
(ii) the disintegrant is selected from the group consisting of croscarmellose sodium, crospovidone, starch, and sodium starch glycolate, or a combination thereof;
(iii) the channeling agent is polyethylene glycol (PEG), sodium chloride, citric acid, sodium citrate, sodium bicarbonate, potassium chloride, potassium citrate, fructose, saccharin sodium, xylitol, or a combination thereof;
(iv) the glidant is selected from the group consisting of silicon dioxide, magnesium stearate, talc, or a combination thereof, and
(v) the lubricant is selected from the group consisting of magnesium stearate, calcium stearate, zinc stearate, sodium stearate, sodium stearyl fumarate, stearic acid, aluminum stearate, leucine, glyceryl behenate, and hydrogenated vegetable oil, or a combination thereof.

129. The composition of claim 128, wherein the diluent or the filler is microcrystalline cellulose (MCC), lactose, mannitol, or a combination thereof.

130. The composition of claim 125, wherein the composition comprises about 15% the compound of Formula (II), or a pharmaceutically acceptable salt thereof, and
(i) about 44% MCC,
(ii) about 14% lactose,
(iii) about 14% sodium chloride,
(iv) about 10% sodium starch glycolate,
(v) about 1% silicon dioxide, and
(vi) about 1% magnesium stearate,
wherein each percentage is a weight percentage by weight of the total composition.

131. The composition of claim 125, wherein the composition comprises about 15% the compound of Formula (II), or a pharmaceutically acceptable salt thereof, and
(i) about 44% MCC,
(ii) about 14% mannitol,
(iii) about 14% sodium chloride,
(iv) about 10% sodium starch glycolate,
(v) about 1% silicon dioxide, and
(vi) about 1% magnesium stearate,
wherein each percentage is a weight percentage by weight of the total composition.

132. The composition of claim 125, wherein the composition comprises about 15% the compound of Formula (II), or a pharmaceutically acceptable salt thereof, and
(i) about 48% MCC,
(ii) about 25% lactose,
(iii) about 5% sodium chloride,
(iv) about 5% sodium starch glycolate,
(v) about 1% silicon dioxide, and
(vi) about 1% magnesium stearate,
wherein each percentage is a weight percentage by weight of the total composition.

133. The composition of claim 125, wherein the composition comprises about 15% the compound of Formula (II), or a pharmaceutically acceptable salt thereof, and
(i) about 58% MCC,
(ii) about 15% lactose,
(iii) about 5% sodium chloride,
(iv) about 5% sodium starch glycolate,
(v) about 1% silicon dioxide, and
(vi) about 1% magnesium stearate,
wherein each percentage is a weight percentage by weight of the total composition.

134. The composition of claim 125, wherein the composition comprises about 7.5% the compound of Formula (II), or a pharmaceutically acceptable salt thereof, and
(i) about 56% MCC,
(ii) about 15% lactose,
(iii) about 15% sodium chloride,
(iv) about 5% sodium starch glycolate,
(v) about 1% silicon dioxide, and
(vi) about 0.5% magnesium stearate,
wherein each percentage is a weight percentage by weight of the total composition.

135. The composition of claim 125, wherein the composition comprises about 6.5% the compound of Formula (II), or a pharmaceutically acceptable salt thereof, and
(i) about 56% MCC,
(ii) about 15% lactose,
(iii) about 15% sodium chloride,
(iv) about 5% sodium starch glycolate,
(v) about 1% silicon dioxide, and
(vi) about 0.5% magnesium stearate,
wherein each percentage is a weight percentage by weight of the total composition.

136. The composition of claim 135, wherein the compound of Formula (II), or a pharmaceutically acceptable salt thereof, is in amount of less than about 50 mg.

137. The composition of claim 136, wherein the compound of Formula (II), or a pharmaceutically acceptable salt thereof, is in an amount of about 5 mg, about 10 mg, about 20 mg, or about 40 mg.

138. The composition of claim 125, wherein the composition is in solid dosage form.

139. The composition of claim 138, wherein the solid dosage form is a capsule, a pill, a cachet, a tablet, a granule, or powder.

140. A method of treating a cholestatic liver disease in a subject in need thereof, wherein the method comprises administering to the subject a therapeutically effective amount of the pharmaceutical composition of claim 125.

141. The method of claim 140, wherein the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.

142. The method of claim 141, wherein the cholestatic liver disease is characterized by pruritus.

143. A kit for treating cholestatic liver disease in a subject in need thereof, wherein the kit comprises at least one unit dosage of a therapeutically effective amount of the composition of claim 125 and instructions for the administration thereof.

144. The kit of claim 143, wherein the cholestatic liver disease is non-obstructive cholestasis, extrahepatic cholestasis, intrahepatic cholestasis, primary intrahepatic cholestasis, secondary intrahepatic cholestasis, progressive familial intrahepatic cholestasis (PFIC), PFIC type 1, PFIC type 2, PFIC type 3, benign recurrent intrahepatic cholestasis (BRIC), BRIC type 1, BRIC type 2, BRIC type 3, total parenteral nutrition associated cholestasis, paraneoplastic cholestasis, Stauffer syndrome, intrahepatic cholestasis of pregnancy (ICP), contraceptive-associated cholestasis, drug-associated cholestasis, infection-associated cholestasis, Dubin-Johnson Syndrome, primary biliary cirrhosis (PBC), primary sclerosing cholangitis (PSC), gallstone disease, Alagille syndrome (ALGS), biliary atresia (BA), post-Kasai biliary atresia, post-liver transplantation biliary atresia, post-liver transplantation cholestasis, post-liver transplantation associated liver disease, intestinal failure associated liver disease, bile acid mediated liver injury, MRP2 deficiency syndrome, or neonatal sclerosing cholangitis.
